(19) United States
(12) Patent Application Publication
(10) Pub. No.: US 2026/0090854 A1
(43) Pub. Date: Apr. 2, 2026

(54) MODULAR CONTROL SHELL

(71) Applicant: Intuitive Surgical Operations, Inc., Sunnyvale, CA (US)

(72) Inventors: Andrej Simeunovic, Sunnyvale, CA (US); Heath Corbet, Sunnyvale, CA (US); Nicole Kernbaum, Sunnyvale, CA (US); Matthew D. Rohr Daniel, Sunnyvale, CA (US); Nicola Diolaiti, Sunnyvale, CA (US)

(73) Assignee: Intuitive Surgical Operations, Inc., Sunnyvale, CA (US)

(21) Appl. No.: 19/346,160

(22) Filed: Sep. 30, 2025

Related U.S. Application Data
(60) Provisional application No. 63/701,358, filed on Sep. 30, 2024.
(60) Provisional application No. 63/701,372, filed on Sep. 30, 2024.

(51) Int. Cl. A61B 34/37; A61B 34/00; A61B 34/20; A61B 34/35

(52) U.S. Cl. CPC A61B 34/37; A61B 34/20; A61B 34/25; A61B 34/35; A61B 34/70; A61B 2034/2051; A61B 2034/2055

(57) ABSTRACT

A control shell, that is an accessory to a surgical instrument control device of a teleoperated surgical system, includes: a body, formed to overlay onto a user input console of the surgical instrument control device, that includes: a cutout region disposed to allow a first preexisting control element of the surgical instrument control device to remain accessible through the control shell to an operator of the surgical instrument control device; a shell control element for controlling a function of the teleoperated surgical system, and a communication interface; a memory that includes instructions for integrating the shell control element into the surgical instrument control device and controlling robotic movement of an instrument; and a processor configured to: communicate with the surgical instrument control device via the communication interface, and integrate the shell control element into the surgical instrument control device based on the instructions stored in the memory.

### CROSS-REFERENCE TO RELATED APPLICATIONS

This application claims the benefit of U.S. Provisional Application No. 63/701,358, filed on Sep. 30, 2024, and U.S. Provisional Application No. 63/701,372, filed on Sep. 30, 2024, which are hereby incorporated by reference herein.

### TECHNICAL FIELD

The present invention generally provides an improved control interface and method of controlling a teleoperated surgical system.

### BACKGROUND

Teleoperated systems can be used to perform a task within a workspace. For example, a teleoperated system may comprise a handheld, motorized tool assembly. As another example, a teleoperated system may comprise a robotic system, and may include one or more robotic manipulators to manipulate instruments for performing the task.

Example teleoperated system include industrial and recreational robotic systems. Example teleoperated systems also include medical robotic systems used in procedures for diagnosis, non-surgical treatment, surgical treatment, etc. A teleoperated surgical system can be automated, semi-automated, teleoperated, etc. An input control console for a teleoperated surgical system may be configured to perform a variety of functions. However, the control capabilities of the input control console are limited by the preexisting control scheme (i.e., the number, n, and arrangement of preexisting control elements) at the time of manufacture.

In this regard, modifying or supplementing the control scheme of the input control console may be useful in expanding the capability of a teleoperated surgical system. For example, a teleoperated surgical system may be upgraded with a new functionality that was not originally included in an original surgical instrument control device. Alternatively, a functionality of the surgical instrument control device may be modified to perform new or different functions based on procedure-specific requirements.

Accordingly, it is desirable to customize an input control console of a teleoperated surgical system to the specific needs of a procedure and/or operator.

### SUMMARY

In general, in one aspect, one or more embodiments relate to a control shell that is an accessory to a surgical instrument control device of a teleoperated surgical system. The control shell includes: a body, formed to overlay onto a user input console of the surgical instrument control device, that includes: a cutout region disposed to allow a first preexisting control element of the surgical instrument control device to remain accessible through the control shell to an operator of the surgical instrument control device; a shell control element for controlling a function of the teleoperated surgical system, and a communication interface; a memory that includes instructions for integrating the shell control element into the surgical instrument control device and controlling robotic movement of an instrument; and a processor configured to: communicate with the surgical instrument control device via the communication interface, and integrate the shell control element into the surgical instrument control device based on the instructions stored in the memory.

In general, in one aspect, one or more embodiments relate to a teleoperated surgical system including: the control shell described above; and the surgical instrument control device.

In general, in one aspect, one or more embodiments relate to a method of operating a teleoperated surgical system including a control shell that is an accessory to a surgical instrument control device, the method comprising: transmitting, from the control shell to the surgical instrument control device, control information (including a correlation between a function of the teleoperated surgical system and a shell control element) corresponding to a shell control element of the control shell; modifying a preexisting control scheme (including instructions for controlling a first robotic movement of a first instrument according to a first user input received at the surgical instrument control device) stored in the preexisting input device to include the control information (including instructions for controlling a second robotic movement of a second instrument according to a second user input received at the shell control element); and initializing the teleoperated surgical system to receive and process both the second user input from the control shell element and the first user input from the surgical instrument control device. The second instrument is configured to perform the first robotic movement. The first instrument is not configured to performing the second robotic movement. The surgical instrument control device is not configured to receive the second user input.

Other aspects of the invention will be apparent from the following description and the appended claims.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be shown and/or labeled in every drawing. A dashed reference numeral indicator line for structural elements indicates the structural element may be partial or fully overlapped (e.g., obscured by a separate structure, covered by a transparent separate structure, indicating a reverse side of a structure). In the drawings:

FIG. 1 is a simplified diagram of a medical system according to some embodiments.

FIG. 2A is a simplified diagram of a medical instrument system according to some embodiments.

FIG. 2B is a simplified diagram of a medical instrument including a manipulator assembly within a flexible elongate device according to some embodiments.

FIG. 3A is a simplified diagram of a side view of a patient coordinate space including a medical instrument according to some embodiments.

FIG. 3B is a simplified perspective diagram of an input control console according to some embodiments.

FIG. 4A is a simplified perspective diagram of a control shell according to some embodiments.

FIG. 4B is a simplified perspective diagram of the control shell of FIG. 4A installed on the input control console of FIG. 3B according to some embodiments.

FIG. 5A is a simplified diagram of a side view of a patient coordinate space including a medical instrument with an additional degree of freedom according to some embodiments.

FIG. 5B is a simplified perspective diagram of an input control console and a control shell according to some embodiments.

FIG. 6A is a simplified diagram of a side view of a shell control element in a first configuration according to some embodiments.

FIG. 6B is a simplified diagram of a side view of the shell control element of FIG. 6A in a second configuration according to some embodiments.

FIGS. 7A-7B are simplified perspective diagrams of a control shell and a surgical instrument control device according to some embodiments.

FIGS. 8A-8B are simplified perspective diagrams of a control shell and a surgical instrument control device according to some embodiments.

FIGS. 9A-9B are simplified perspective diagrams of a control shell and a surgical instrument control device according to some embodiments.

FIG. 10A is a simplified schematic of a medical instrument system according to some embodiments.

FIG. 10B is a simplified schematic of the medical instrument system of FIG. 10A with a separate system according to some embodiments.

FIG. 11A is a simplified schematic of a medical instrument system with an separate fluid input device according to some embodiments.

FIG. 11B is a simplified perspective diagram of a control shell installed on an input control console for the medical instrument system of FIG. 11A according to some embodiments.

FIGS. 11C-11D are simplified perspective diagrams of an alternate control shell installed on an input control console for the medical instrument system of FIG. 11A according to some embodiments.

FIG. 11E is a simplified perspective diagrams of an external portion of the alternate control shell of FIG. 11C according to some embodiments.

FIG. 11F is a simplified perspective diagrams of an internal portion of the alternate control shell of FIG. 11C according to some embodiments.

FIG. 12 is simplified perspective diagram of another control shell installed on the input control console for the medical instrument system of FIG. 11A according to some embodiments.

FIG. 13 is a schematic diagram that shows an example of a computing system according to some embodiments.

FIG. 14 is a flowchart of a method of operating a teleoperated surgical system according to some embodiments.

FIG. 15A is a flowchart of a method of operating a teleoperated surgical system according to some embodiments.

FIG. 15B is a communication diagram corresponding to the method of FIG. 15A according to some embodiments.

FIG. 16A is a flowchart of a method of operating a teleoperated surgical system according to some embodiments.

FIG. 16B-16C are communication diagrams corresponding to the method of FIG. 16A according to some embodiments.

FIG. 17A is a flowchart of a method of operating a teleoperated surgical system according to some embodiments.

FIG. 17B is a communication diagram corresponding to the method of FIG. 17A according to some embodiments.

FIG. 18 is a flowchart of a method of operating a teleoperated surgical system according to some embodiments.

FIG. 19 is a flowchart of a method of operating a teleoperated surgical system according to some embodiments.

FIG. 20A is a flowchart of a method of operating a teleoperated surgical system according to some embodiments.

FIG. 20B-20D are communication diagrams corresponding to the method of FIG. 20A according to some embodiments.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements, and is not to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

This disclosure describes various devices, elements, and portions of computer-assisted systems (e.g., a teleoperated surgical system) and elements in terms of their state in three-dimensional space. As used herein, the term “position” refers to the location of an element or a portion of an element (e.g., three degrees of translational freedom in a three-dimensional space, such as along Cartesian x-, y-, and z-coordinates). As used herein, the term “orientation” refers to the rotational placement of an element or a portion of an element (e.g., three degrees of rotational freedom in three-dimensional space, such as about roll, pitch, and yaw axes, represented in angle-axis, rotation matrix, quaternion representation, and/or the like). As used herein, and for a device with a kinematic series, such as with a repositionable structure with a plurality of links coupled by one or more joints, the term “proximal” refers to a direction toward a base of the kinematic series, and “distal” refers to a direction away from the base along the kinematic series.

As used herein, the term “pose” refers to the multi-degree of freedom (DOF) spatial position and orientation of a coordinate system of interest attached to a rigid body. In general, a pose includes a pose variable for each of the DOFs in the pose. For example, a full 6-DOF pose for a rigid body in three-dimensional space would include 6 pose variables corresponding to the 3 positional DOFs (e.g., x, y, and z) and the 3 orientational DOFs (e.g., roll, pitch, and yaw). A 3-DOF position only pose would include only pose variables for the 3 positional DOFs. Similarly, a 3-DOF orientation only pose would include only pose variables for the 3 rotational DOFs. Further, a velocity of the pose captures the change in pose over time (e.g., a first derivative of the pose). For a full 6-DOF pose of a rigid body in three-dimensional space, the velocity would include 3 translational velocities and 3 rotational velocities. Poses with other numbers of DOFs would have a corresponding number of velocities translational and/or rotational velocities.

Aspects of this disclosure are described in reference to a modular control shell that modifies the capabilities of a surgical instrument control device of a teleoperated surgical system. A teleoperated surgical system may include devices (e.g., a manipulator assembly, an auxiliary system that is separate from but in coordination with a manipulator system) that are teleoperated, externally manipulated, autonomous, semiautonomous, and/or the like. In one or more embodiments, a manipulator assembly is implemented using a teleoperated surgical system, such as the Ion® System commercialized by Intuitive Surgical, Inc. of Sunnyvale, California. Knowledgeable persons will understand, however, that inventive aspects disclosed herein may be embodied and implemented in various ways, including teleoperated and non-teleoperated, and medical and non-medical embodiments and implementations. Implementations on Ion® Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for simulating humans, animals, portions of human or animal anatomy, industrial systems, general robotic, or teleoperated systems. As further examples, the models, instruments, systems, and methods described herein may be used for simulating non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like.

In some embodiments, the control shell supplements or modifies a control console (e.g., an input control console, a surgical instrument control device) of a teleoperated surgical system, such as the Ion® System. The control console includes a preexisting control scheme with a fixed number of controls corresponding to the degrees of freedom (DOF) of the manipulator assembly. Accordingly, the preexisting control scheme includes instructions for controlling the teleoperated surgical system based on interaction with control elements of the control console. In some embodiments, the preexisting control scheme includes instructions for controlling a first robotic movement of a first instrument (e.g., a flexible elongate device) according to a first user input received at the surgical instrument control device. For example, a control console may include a trackball for controlling a flexible elongate device, a scroll wheel for controlling insertion of the flexible elongate device, and one or more buttons to control actions performed by the flexible elongate device or an instrument attached thereon. In instances where a new manipulator assembly is required to perform a procedure (e.g., the preexisting system is supplemented with additional support equipment, a new DOF, such as instrument operation or roll motion, is introduced), the preexisting control scheme of the control console will not provide an operator with a sufficient number of controls or control information to operate the new manipulator assembly. In some instances, a new manipulator assembly benefits or requires displaying additional control information or modifying existing display control information, such as position or actuation information.

Accordingly, in one or more embodiments, a control shell is configured to directly attach to the control console and provide a new control scheme. For example, when the preexisting control scheme includes three DOF (e.g., an insertion axis, controlled by a scroll wheel, and two directions of flexure in the flexible elongate device, controlled by a trackball), the control shell may include a new control for an additional DOF (e.g., a scrolling knob to control a roll axis of the flexible elongate device) to supplement the three preexisting DOF.

Similarly, any other control elements (e.g., buttons, switches) of the preexisting control scheme maybe be supplemented by additional elements on the control shell.

In some embodiments, the control shell may include a new control scheme that reorganizes the layout of the preexisting controls. Furthermore, the control shell may be configured to modify the preexisting control scheme by disabling (hardware lock (e.g., keyed control element, physically covering or obscuring the preexisting control element) or software lock (e.g., disabling preexisting control software)) or changing the behavior (e.g., modifying the preexisting control software) of the preexisting DOF or control elements.

A more detailed discussion of various embodiments is provided below in reference to the figures.

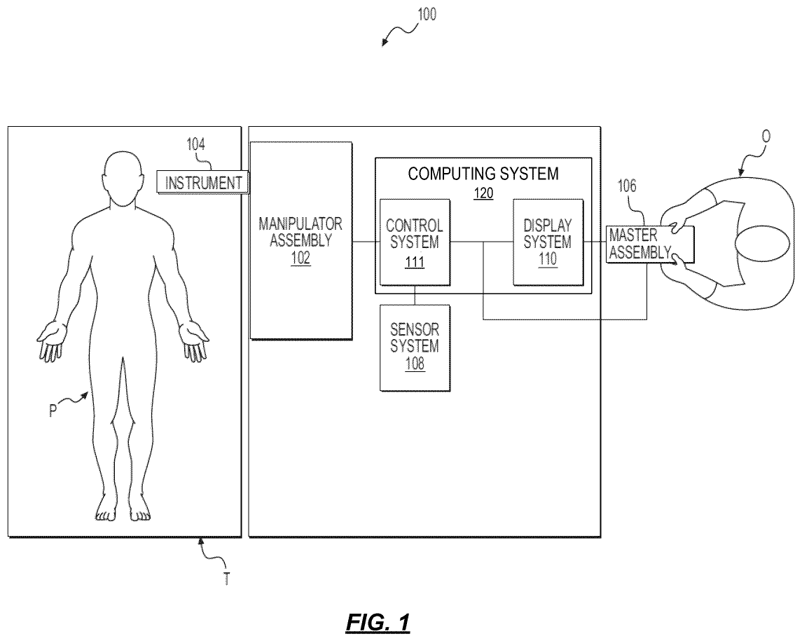

FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, a teleoperated surgical system (e.g., medical system 100) may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. The manipulator assembly 102 may be mounted to and/or positioned near a patient table T. A master assembly 106 allows an operator O (e.g., a surgeon, a clinician, a physician, or other operator) to control the manipulator assembly 102. In some examples, the master assembly 106 allows the operator O to view the procedural site or other graphical or informational displays. In some examples, the manipulator assembly 102 may be excluded from the medical system 100 and the medical instrument 104 may be controlled directly by the operator O. In some examples, the manipulator assembly 102 may be manually controlled by the operator O.

Direct operator control may include various handles and operator interfaces for hand-held operation of the medical instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T.

The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo-controlled links (e.g., one or more links that may be manually positioned and locked in place) and/or one or more servo-controlled links (e.g., one or more links that may be controlled in response to commands, such as from an input device 112 (i.e., a preexisting input device, a surgical instrument control device)). The manipulator assembly 102 may include a plurality of actuators (e.g., motors) that drive inputs on the medical instrument 104 in response to commands, such as from the input device 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument 104 about an axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site.

In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the input device 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 111, which may include processing circuitry that implements the some or all of the methods or functionality discussed herein. The control system 111 may include at least one memory and at least one processor for controlling the operations of the manipulator assembly 102, the medical instrument 104, the master assembly 106, the sensor system 108, and/or the display system 110. Control system 111 may include instructions (e.g., a non-transitory machine-readable medium storing the instructions) that when executed by the at least one processor, configures the one or more processors to implement some or all of the methods or functionality discussed herein. While the control system 111 is shown as a single block in FIG. 1, the control system 111 may include two or more separate data processing circuits with one portion of the processing being performed at the manipulator assembly 102, another portion of the processing being performed at the master assembly 106, and/or the like. In some examples, the control system 111 may include other types of processing circuitry, such as application-specific integrated circuits (ASICs) and/or field-programmable gate array (FPGAs). The control system 111 may be implemented using hardware, firmware, software, or a combination thereof.

In some examples, the control system 111 may receive feedback from the medical instrument 104, such as force and/or torque feedback. Responsive to the feedback, the control system 111 may transmit signals to the master assembly 106. In some examples, the control system 111 may transmit signals instructing one or more actuators of the manipulator assembly 102 to move the medical instrument 104. In some examples, the control system 111 may transmit informational displays regarding the feedback to the display system 110 for presentation or perform other types of actions based on the feedback.

The control system 111 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 111 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016, and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011, and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

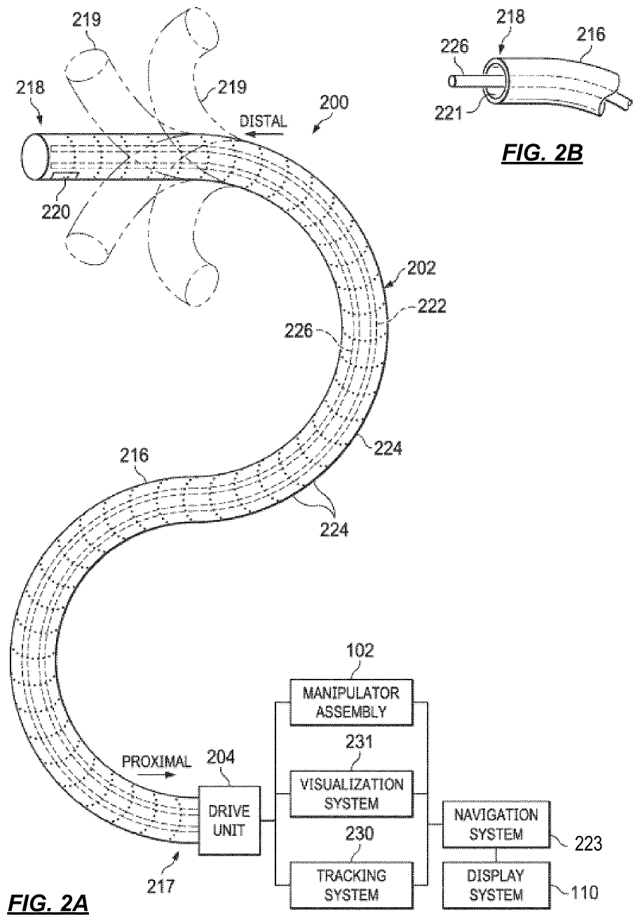

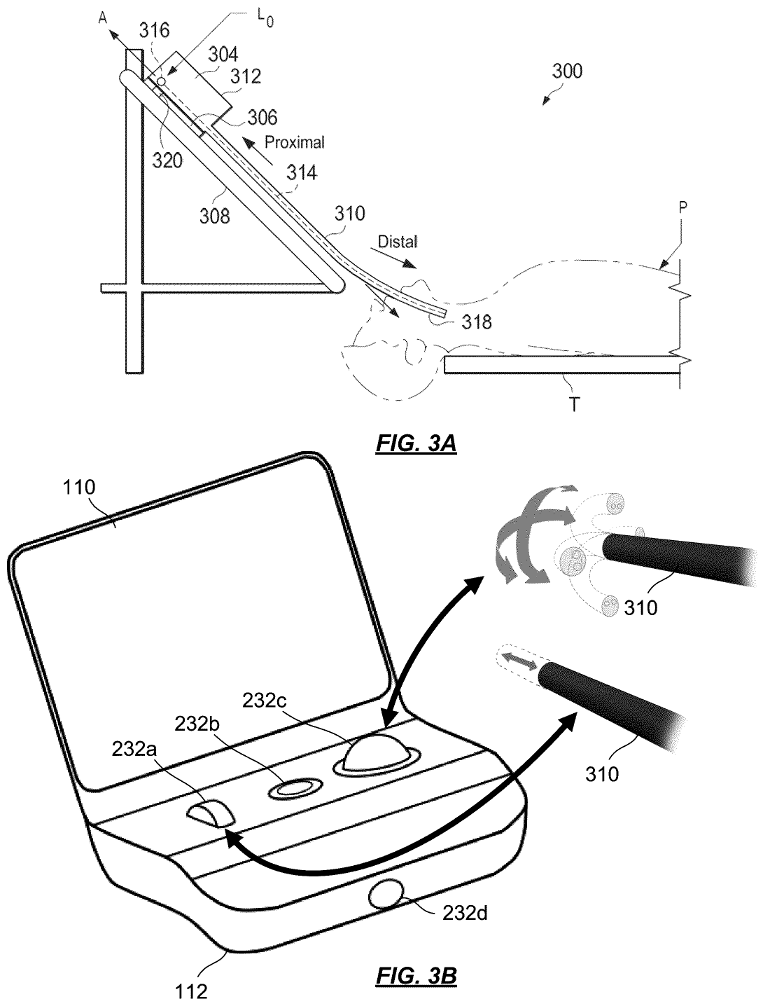

FIG. 2A is a simplified diagram of a medical instrument system 200 according to some embodiments.

The medical instrument system 200 includes a flexible elongate device 202 (also referred to as elongate device 202), a drive unit 204, and a flexible tool 226 (e.g., a medical tool) that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, tool recognition sensor 233, and navigation system 223 are also shown in FIG. 2A and are example components of the control system 111 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel or lumen 221 through which a flexible tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 111 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexible body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sept. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999, and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor system 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the flexible tool 226 within the elongate device 202 according to some embodiments.

The flexible body 216 of the elongate device 202 may include the lumen 221 sized and shaped to receive the flexible tool 226. In some embodiments, the flexible tool 226 may be used for procedures such as diagnostics, imaging, surgery, biopsy, ablation, illumination, irrigation, suction, electroporation, etc. Flexible tool 226 can be deployed through channel or lumen 221 of flexible body 216 and operated at a procedural site within the anatomy. Flexible tool 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the flexible tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The flexible tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the lumen 221 when the biopsy tool is within the lumen 221. The flexible tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of flexible tool 226 to facilitate simultaneous image capture and tissue intervention, such as within the same lumen 221 or in separate channels. A flexible tool 226 may be advanced from the opening of the lumen 221 to perform the procedure (or some other functionality) and then retracted back into the lumen 221 when the procedure is complete. The flexible tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 216 may include one or more dedicated channels that carry the cable(s) and/or optical fiber(s) between the distal end 218 and the visualization system 231.

Here, the medical instrument system 200 can perform simultaneous imaging and tool operations.

In some examples, the medical tool 226 is capable of controllable articulation.

The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005, and titled “Articulated Surgical Instrument for Performing Minimally Invasive Surgery with Enhanced Dexterity and Sensitivity”) and U.S. Pat. No. 9,259,274 (filed Sept. 30, 2008, and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019, and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. The drive unit 204 may further include brakes.

One brake may be paired with one actuator. In configurations that pair an actuator with a gear reducer, the brake may be located on the actuator side, which enables even a relatively small brake to produce a significant braking force. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 223, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 223 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011, and titled “Medical System Providing Dynamic Registration of a Model of an Anatomic Structure for Image-Guided Surgery”), which is incorporated by reference herein in its entirety.

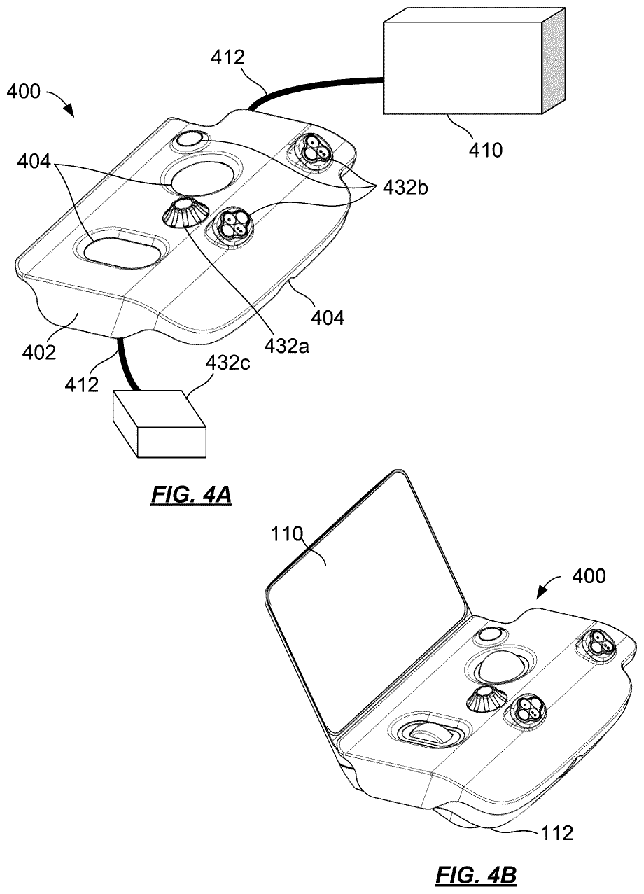

FIG. 3A is a simplified diagram of a side view of a patient coordinate space including a medical instrument 304 according to some embodiments.

As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue.

Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device).

The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308. The instrument carriage 306 or insertion stage 308 may further include brakes. One brake may be paired with one actuator. For example, an actuator may be provided for driving the medical instrument along the insertion axis of the manipulator assembly, and a brake may be provided for inhibiting movement of the medical instrument along the insertion axis.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIG. 3A. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position L0 on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0).

FIG. 3B is a simplified perspective diagram of an surgical instrument control device 112 according to some embodiments.

The surgical instrument control device 112 may correspond to or may be part of the master assembly 106. A top surface of surgical instrument control device 112 includes various input controls such as, for example an insertion/retraction control 232a, a passive control button 232b, a steering control 232c, and an emergency stop button 232d. The input control console may further include an integrated display screen (e.g., part of the display system 110).

Although FIG. 3B shows a configuration of the various input controls for an elongate device, it should be understood that surgical instrument control device 112 can control any variety of instruments and devices and the exact placement, orientation, relative-positioning, and/or the like of the various input controls are exemplary only. It is understood that other configurations of input controls, different numbers of input controls, and/or the like are possible. In some embodiments, surgical instrument control device 112 is suitable for use as a patient-side input control unit for the elongate device and may, for example, be mounted in proximity to insertion stage 308.

Although not shown in FIG. 3B, surgical instrument control device 112 may optionally include one or more circuit boards, logic boards, and/or the like that are usable to provide power, signal conditioning, interface, and/or other circuitry for surgical instrument control device 112. In some examples, the one or more circuit boards, logic boards, and/or the like are useable to interface surgical instrument control device 112 and its various input controls to a control unit for the elongate device. In some examples, the control unit of the elongate device corresponds to the control device of master assembly 106 and/or the like. In some examples, the one or more circuit boards, logic boards, and/or the like may include memory and one or more one or more processors, multi-core processors, field programmable gate arrays (FPGAs), application specific integrated circuits (ASICs), and/or the like. In some examples, the memory may include one or more types of machine-readable media. Some common forms of machine-readable media may include floppy disk, flexible disk, hard disk, magnetic tape, any other magnetic medium, CD-ROM, any other optical medium, RAM, PROM, EPROM, FLASH-EPROM, any other memory chip or cartridge, and/or any other medium from which a processor or computer is adapted to read.

In some examples, insertion/retraction control 232a is a single DOF with infinite length of travel input control providing infinite length of travel along a first axis usable by the operator to control the insertion depth of the distal end of the elongate device.

Insertion/retraction control 232a is depicted as a scroll wheel, however, other types of input controls, including non-infinite length of travel input controls (e.g., a finite travel-length slider) and/or a velocity-based input controls (e.g., a joystick), are possible. In some examples, scrolling of the scroll wheel forward away from the operator increases the insertion depth (insertion) of the distal end of the elongate device and scrolling of the scroll wheel backward toward the operator decreased the insertion depth (retraction) of the distal end of the elongate device. In some examples, insertion/retraction control 232a is usable by the operator to move instrument carriage 306 in and out along insertion stage 308 in order to control the insertion depth of distal end 318.

When insertion/retraction control 232a is an infinite length of travel input control, operating insertion/retraction control 232a in a position-specifying mode allows the operator to exercise precise insertion depth control of the distal end of the elongate device over the full length of travel of the elongate device. In some examples, movement of insertion/retraction control 232a may be detected by the one or more circuit boards, logic boards, and/or the like of surgical instrument control device 112 using one or more encoders, resolvers, optical sensors, hall effect sensors, and/or the like (not shown). In some examples, feedback applied via one or more electromagnetic actuators, and/or the like may optionally be used to apply haptic feedback to insertion/retraction control 232a. In some examples, a scale factor between an amount of movement of insertion/retraction control 232a and an amount of insertion and/or retraction movement by the elongate device is adjustable by the operator and/or control software of the elongate device so that an insertion/retraction velocity of the elongate device relative to an angular velocity of insertion/retraction control may be adjusted to allow both fast insertion and retraction when advantageous and slower more precise insertion and retraction when greater control precision is desired.

In some examples, passive control button 232b is a single DOF with binary output single. While the drawings depict the passive control button 232b as a button or collection of buttons, it will be appreciated that other interfaces that respond to a presence of the operator or an interaction from the operator are possible. For example, an interface may include any number of buttons, toggle switches, capacitive switches, a latching or non-latching control element, or any combination thereof. Furthermore, an interface may include capacitive or inductive sensors that enable touch-based interaction modalities. In some examples, a collection of passive control elements may be grouped onto a single panel. In some embodiments, the passive control button 232b may be combined with another control element (e.g., a button on a joystick).

In some examples, steering control 232c is a multi-degree of freedom infinite length of travel input control providing infinite length of travel about any number of axes, which in practice may be decomposed into combinations of a left and right rotation, a forward and back rotation, and a spin in place rotation. While steering control 232c is depicted as a track ball, other types of input controls, including non-infinite length of travel input controls (e.g., one or more finite travel-length sliders) and/or a velocity-based input controls (e.g., a joystick), are possible.

Steering control 232c is usable by the operator to concurrently control both the pitch and yaw of the distal end of the elongate device. In some examples, components of the track ball rotation in the forward and back directions may be used to control a pitch of the distal end of the elongate device and components of the track ball rotation in the left and right directions may be used to control a yaw of the distal end of the elongate device. In some examples, other rotational components of the track ball may be used to control pitch and/or yaw with the operator being optionally able to control whether the direction of rotation is normal and/or inverted relative to the direction applied to the steering (e.g., rotate forward to pitch down and backward to pitch up versus backward to pitch down and forward to pitch up). In some embodiments, a rotational component of the track ball (e.g., a 3rd DOF relative to the pitch DOF and yaw DOF) may be used to control a roll movement of the elongate device). In some examples, steering control 232c is usable by the operator to manipulate the distances each of the cables extending between the proximal and distal ends of the elongate device are pushed and/or pulled.

In some embodiments, insertion/retraction control 232a, passive button control 232b, and/or steering control 232c include a touch sensor. The touch sensor may be a capacitive touch sensor, any other type of touch sensor (e.g., a luminosity sensor that detects reflected or scattered light, a contact sensor, a distance sensor), or any combination of sensors.

Alternatively or additionally, a pressure sensor may be included. The touch and/or pressure sensor at the surgical instrument control device 112 may be used to differentiate intended movement by the operator from inadvertent movement due to accidental contact, dropping of surgical instrument control device 112, and/or the like. For example, the control shell 400 may be configured to disable or enable a function of the control shell 400 (e.g., prevent input from the shell control element 432a or any/all other shell control element(s)) and/or the surgical instrument control device 112 (e.g., prevent input from one or more preexisting control elements of the surgical instrument control device 112) based on a signal from the touch sensor. Other types of proximity sensors (e.g., ultrasonic sensors, vision sensors, light walls, and/or the like) may be used to detect operator proximity to the input controls. In some examples, one or more wrist detection sensors (e.g., capacitive touch, pressure, and/or similar sensors) in a wrist rest may be used to detect operator proximity to the input controls.

FIG. 4A is a simplified perspective diagram of a control shell 400 according to some embodiments.

A control shell 400 is configured to modify the capabilities of a surgical instrument control device 112. Accordingly, the control shell 400 includes various elements and features relating to interfacing with the surgical instrument control device 112, each of which is described in further detail below.

The control shell 400 includes a body 402 that can be formed to fully or at least partially conform to a surface of the surgical instrument control device 112 (e.g., the control console of an Ion® System, a user input console of a surgical instrument control device). In some embodiments, the body 402 can cover only a portion of the surface of the input device, for example, less than 25 % of the surface area. In the non-limiting example shown in FIG. 4A, the body 402 includes a bottom surface that fully or partially conforms to a top surface of the surgical instrument control device 112. Put another way, the body 402 can be configured as a shell that nests over the surgical instrument control device 112. In some embodiments, the body 402 can integrate as an overlay so that the general shape of the surgical instrument control device 112 is disturbed as little as possible so as to provide a user interface that is nearly identical to that of the surgical instrument control device 112. A top surface of the body 402 may be configured to replicate portions of the top surface of the surgical instrument control device 112 (e.g., maintain a consistent layout of labels, indicators, etc.). In some embodiments, the body 402 can include one or more keying features at different locations, generally keying at opposing locations of the body 402, that prevent movement of the body 402 after placement onto input device 112. Such keying features can include portions that extend downward past the top surface of the input device 112, such as lateral edges, lips, contours, or bumpers. In some embodiments, the body 402 can include mechanical attachment elements, such as hand turned knobs/screws, magnets, clasps, suction cups, or straps to prevent movement of the body 402. Generally, the control shell 400 is configured as an accessory to the preexisting input device, and therefore can be easily removed when not required to operate a particular instrument. However, in some embodiments, the control shell 400 can be irremovably attached as a permanent upgrade to the surgical instrument control device 112 (e.g., using a bonding agent or machine screws, in cases where the surgical instrument control device 112 would otherwise be obsolete or provide less clinical value without the control shell 400).

In some embodiments, the top surface may be configured to deviate from the design of the surgical instrument control device 112 to emphasize a different control function that is specific to the control shell. For example, the body 402 may change the layout of labels, indicators, etc. to emphasize a different control scheme. In some embodiments, the body 402 may include a cover (e.g., a flap, a case, a trigger guard, a protrusion) that obstructs a control element on the control shell or on the surgical instrument control device 112 to prevent the operator from using said control element without consciously addressing the cover.

Alternatively, the body 402 may include a fixed cover that completely obstructs a control element and prevents the operator from using the control element (e.g., use is prohibited for certain operators (e.g., a trainee), control prohibited for a specific procedure).

In some embodiments, the body 402 of the control shell 400 is configured to include additional structural features that are specific to a given procedure. For example, the body 402 may include a holster configured to hold one or more medical instruments that can be attached to the manipulator assembly 102. In some embodiments, the holster may be a protrusion on one side of the body 402 that an instrument can fasten or otherwise attach to. In some embodiments, the additional structural features may include a user feedback system (e.g., a lighting system (e.g., one or more lighting elements configured to correspond to specific control elements), a sound device (e.g., a speaker), a haptic feedback device (e.g., a vibration device), etc.) and/or capacitive touch or proximity sensors.

The control shell 400 may include one or more cutout regions 404 in the body 402 that allow one or more preexisting control elements of the surgical instrument control device 112 to be accessible through the body 402 (e.g., protrude through the control shell 400). Each cutout region 404 may be of any size or shape and may conform to the geometry of a preexisting control elements of the surgical instrument control device 112. In some embodiments, the cutout region 404 extends entirely through the body 402 of the control shell 400 (i.e., a through hole). Alternatively, the cutout region 404 may be a constricted portion of the body 402 (e.g., a thin flexible portion of the body 402 (e.g., a membrane)) that allows access to an underlying control element while maintaining a continuous top surface of the control shell 400 (e.g., for ease of cleaning).

The control shell 400 includes a communication interface that is configured to connect to a corresponding communication interface (not shown) of the surgical instrument control device 112. In some embodiments, the communication interface includes connectors for a specific preexisting input device (e.g., Ion® System). In some embodiments, the communication interface includes any appropriate interface (e.g., a CAN interface, an ethernet interface, a universal serial bus (USB) interface, etc.) to facilitate connection to a surgical instrument control device 112. In some embodiments, the communication interface may be configured to connect to a network (not shown) (e.g., a local area network (LAN), a wide area network (WAN) such as the Internet, mobile network, or any other type of network) and/or to another device, such as another computing system. The control shell 400 further includes hardware (e.g., processor and memory), firmware, software, or any combination thereof for managing the communication interface and the connection to the surgical instrument control device 112, as discussed in further detail below.

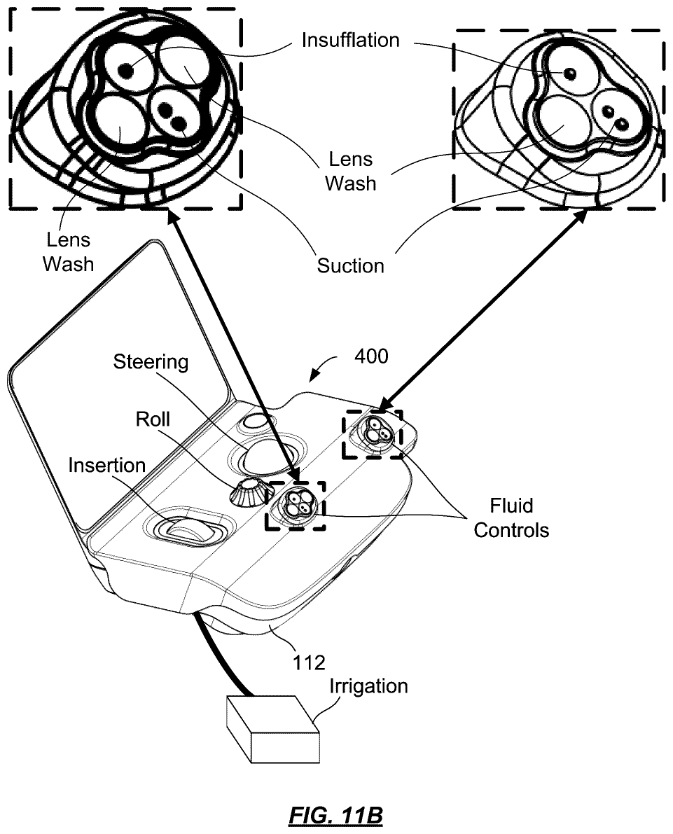

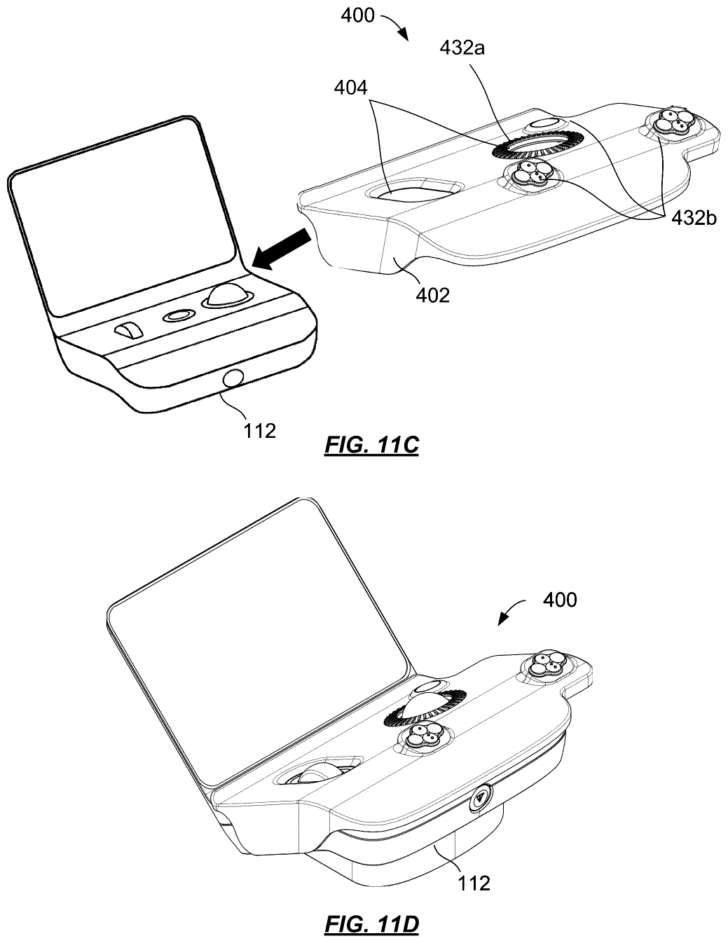

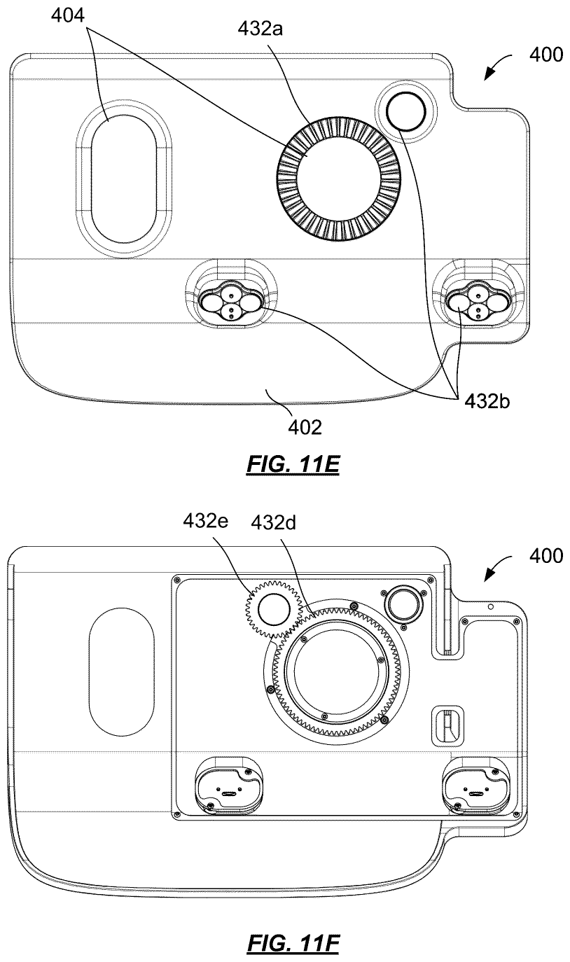

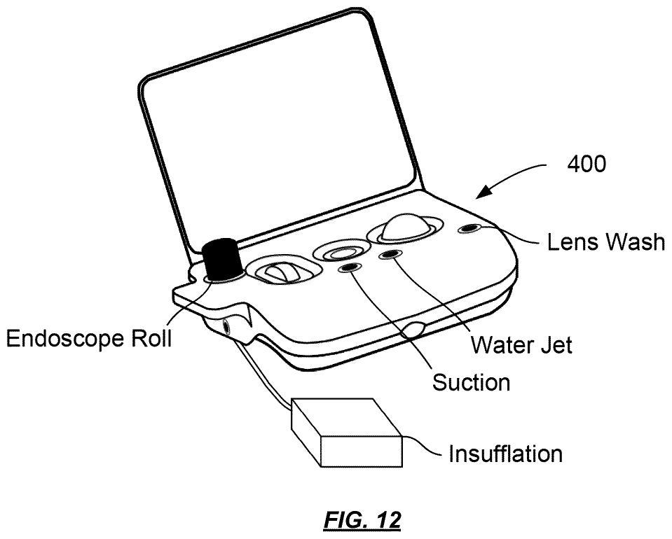

The communication interface of the control shell may also be configured to electrically connect to and/or communicate with a system that is separate from the manipulator assembly and that operates in coordination with the manipulator assembly. In some embodiments, the communication interface includes any appropriate interface to facilitate direct or indirect connection with the separate system. While some embodiments described herein relate to a control shell 400 that directly connects/communicates with a separate system or controller of a separate system (e.g., see FIG. 11A), it will be appreciated that other embodiments may be conceived without deviating from the gist of the disclosure. In some embodiments, the control shell 400 may electrically connect to and/or communicate with the separate system in an indirect manner. For example, the control shell 400 may connect to the preexisting input device, which then relays signals from the control shell 400 to the separate system via new or preexisting channels/interfaces.

In some embodiments, the control shell 400 may include a shell display system 410. As shown in FIG. 3B, the surgical instrument control device 112 may include a display 110. The shell display system 410 is configured to supplement information provided by the display 110 of the surgical instrument control device 112. The control shell 400 further includes hardware (e.g., processor and memory), firmware, software, or any combination thereof for managing the shell display system 410 and, optionally, reconfiguring the display 110 of the surgical instrument control device 112, as discussed in further detail below.

In some embodiments, the shell display system 410 includes a secondary display (e.g., a liquid crystal display (LCD), a plasma display, touchscreen, organic LED display (OLED), projector, or other display device). In some embodiments, the shell display system 410 may include a headset display (e.g., virtual reality (VR), augmented reality (AR), mixed reality (MR) headset). In some embodiments, the shell display system 410 may be integrated into the body 402 of the control shell 400. Alternatively, the shell display system 410 may be separated from the control shell 400 (e.g., operated by wired or wireless connection). Many different types of display elements/systems exist, and the shell display system 410 may take other forms.

The control shell 400 may include one or more connection interfaces 412. A connection interface is a wired or wireless connection to a subcomponent of the control shell 400 (e.g., a removable display system 410, a detachable instrument 104, a control element 432).

The control shell 400 includes one or more shell control elements 432.

Various embodiments of shell control element 432 are described below with respect to FIGS. 4A-9B. However, it will be appreciated that these embodiments are non-limiting and a control shell 400 may include any number or combination of shell control elements 432. The control shell 400 further includes hardware (e.g., processor and memory), firmware, software, or any combination thereof for managing the shell control elements 432 and the corresponding interactions with the surgical instrument control device 112, as discussed in further detail below.

As shown in FIG. 4A, shell control element 432a is a scrolling knob (e.g., infinite length of travel, non-infinite length of travel input control). The shell control element 432a may be configured to control a single new DOF of the manipulator assembly 102 that is not controlled by the surgical instrument control device 112 (e.g., a roll DOF added to the elongate device 202). In some embodiments, a shell control element 432 (e.g., a trackball) may be configured to control multiple DOF (e.g., accessible DOF of the surgical instrument control device 112, new DOF not controlled by the surgical instrument control device 112).

As shown in FIG. 4A, shell control element 432b may include one or more buttons (e.g., an individual button, a collection of multiple buttons, multiple collections of multiple buttons, or any combination thereof). The shell control element 432b may be configured as a passive control input that causes execution of one or more actions by the surgical instrument control device 112 (e.g., a new subroutine, a procedure-specific set of actions, an operator-specific set of actions). In some embodiments, the shell control element 432b may be configured with multiple subroutines based on an input interaction from the operator (e.g., press, release, press-and-hold, multi-press).

As shown in FIG. 4A, shell control element 432c is a foot petal. The shell control element 432c configured to provide the operator with an additional hands-free control input for the surgical instrument control device 112. In some embodiments, the shell control element 432c may be configured to accept another form of hands-free input from the operator (e.g., microphone for voice control, camera for gesture control).

In some embodiments, a shell control element 432 duplicates a function of a preexisting control element with different level of sensitivity. For example, the shell control element 432 may have a different gain setting for translating motion of a scroll wheel or knob to a corresponding movement in the manipulator assembly 102.

While FIG. 4A shows one configuration of the control shell 400, it will be appreciated that other embodiments may be conceived with any number or combination of features without deviating from the gist of the disclosure. Furthermore, while the drawings of throughout the disclosure show a control shell 400 configured for an Ion® System, it will be appreciated that other embodiments may be conceived without deviating from the gist of the disclosure. The control shell 400 may take any appropriate form to attach to any surgical instrument control device 112 of a manipulator assembly 102.

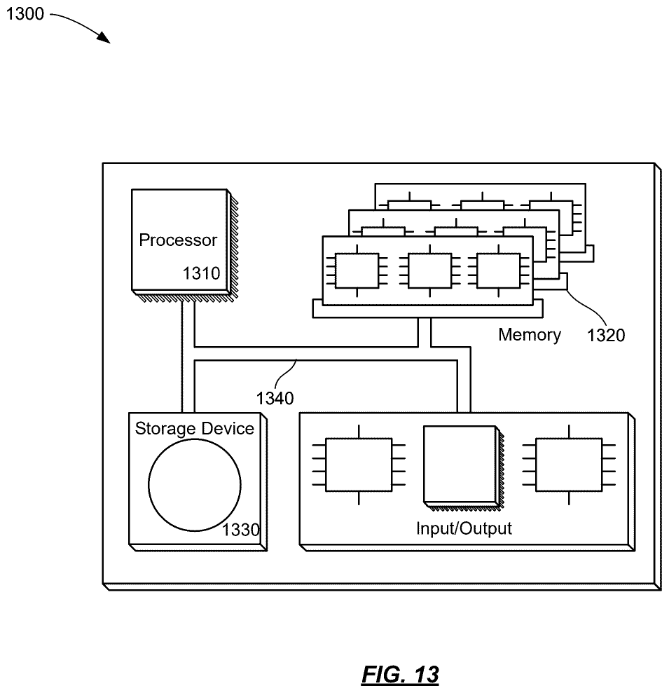

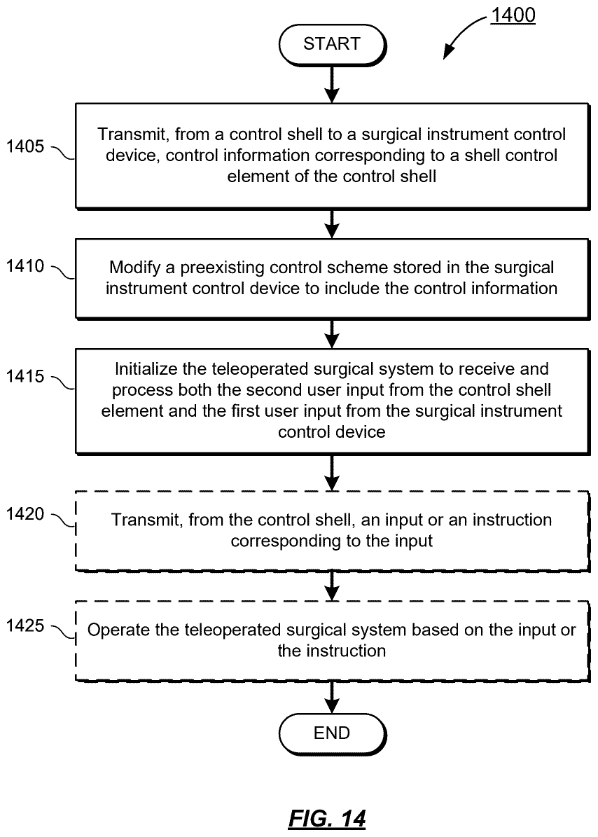

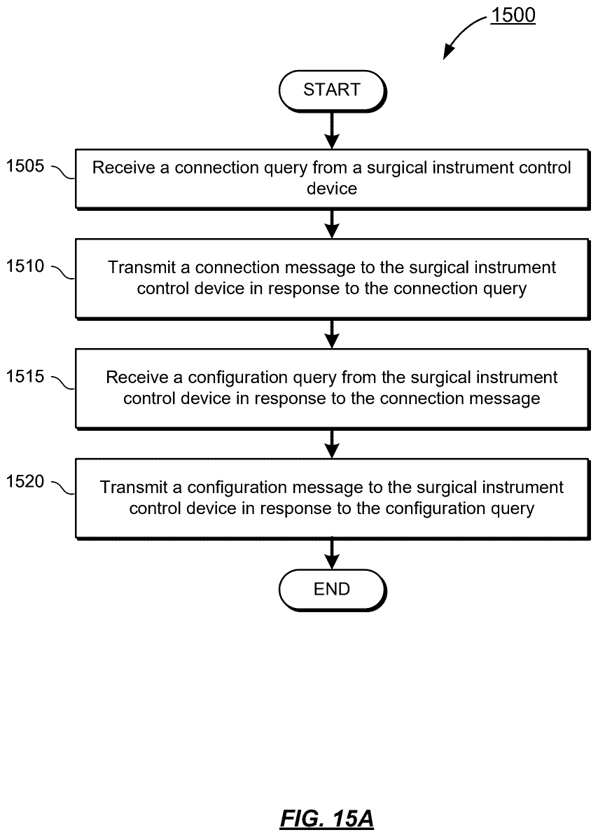

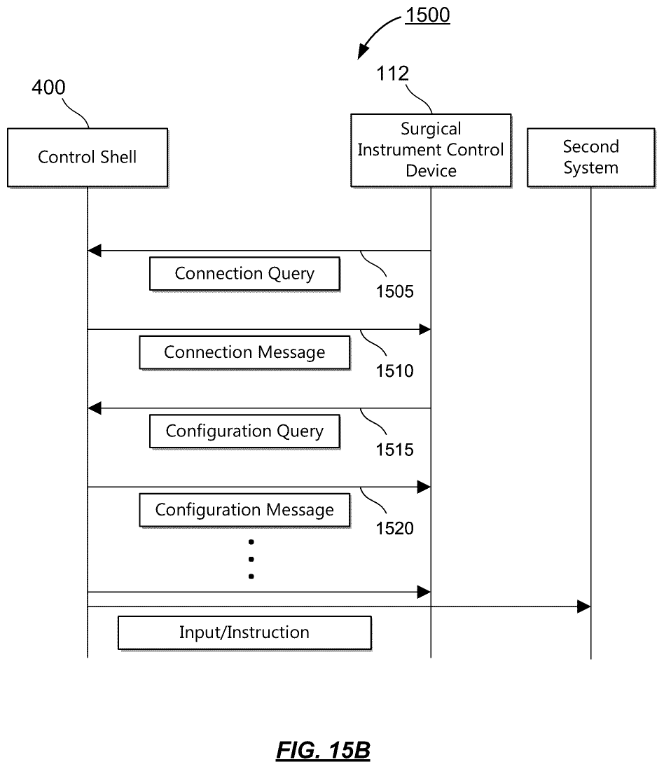

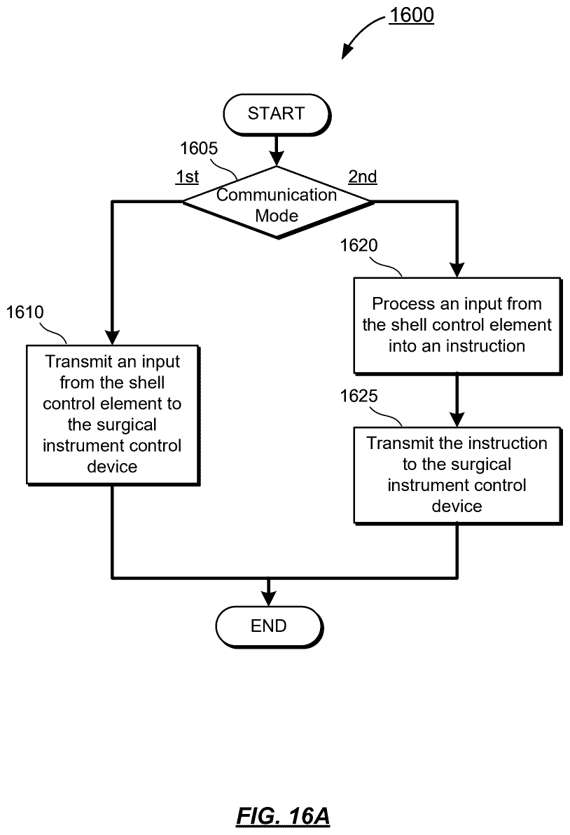

The control shell 400 may include a computing system with one or more computer processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities. A computing system according to some embodiments is described in further detail below with respect to FIG. 13.

As discussed above, although not shown in FIG. 4A, the control shell 400 includes one or more circuit boards, logic boards, and/or the like that are usable to provide power, signal conditioning, interface, and/or other functionality the control shell 400. In some examples, the one or more circuit boards, logic boards, and/or the like are useable to interface the control shell 400 to the surgical instrument control device 112 of an elongate device 202. The memory may include instructions for integrating the shell control element 432 into the surgical instrument control device 112. The processor may be configured to: communicate with the surgical instrument control device 112 via the communication interface and integrate the shell control element 432 into the surgical instrument control device 112 based on the instructions stored in the memory. In some examples, the surgical instrument control device 112 of the elongate device 202 corresponds to the control device of master assembly 106 and/or the like.

In some examples, the one or more circuit boards, logic boards, and/or the like may include memory and one or more one or more processors, multi-core processors, field programmable gate arrays (FPGAs), application specific integrated circuits (ASICs), and/or the like. In some examples, the memory may include one or more types of machine-readable media. Some common forms of machine-readable media may include floppy disk, flexible disk, hard disk, magnetic tape, any other magnetic medium, CD-ROM, any other optical medium, RAM, PROM, EPROM, FLASH-EPROM, any other memory chip or cartridge, and/or any other medium from which a processor or computer is adapted to read.

FIG. 4B is a simplified perspective diagram of the control shell 400 of FIG. 4A installed on the surgical instrument control device 112 of FIG. 3B according to some embodiments.

As discussed above, the geometry of the body 402 of the control shell 400 aligns with and conforms to the surgical instrument control device 112. Furthermore, the cutout regions 404 of the control shell 400 are aligned with the control elements 232a, c, d, such that each of the preexisting control elements 232a, c, d remains accessible to the operator. While preexisting control element 232b (i.e., the central passive button control element) is covered by the body 402 of the control shell 400, a shell control element 432b (e.g., one of the passive button control elements) on the control shell 400 may be configured to replace (i.e., duplicate) or augment the functionality of the preexisting control element 232b. Furthermore, the control shell 400 is configured to match the control surface of the surgical instrument control device 112 without obstructing the display 110. In addition, the control shell 400 supplements the control scheme of the surgical instrument control device 112 with the addition of shell control elements 432.

While the disclosure describes the control shell 400 as substantially recreating the top surface of surgical instrument control device 112, it will be appreciated that other embodiments may be conceived without deviating from the gist of the disclosure. The control shell 400 may take any appropriate shape that integrates with any portion of the surgical instrument control device 112. For example, the control shell 400 may include a single large cutout region 404 that leaves the entire surgical instrument control device 112 accessible to the operator while the body 402 and the shell control element 432 are offset to one side of the surgical instrument control device 112 (e.g., expanding the top surface of the surgical instrument control device 112).

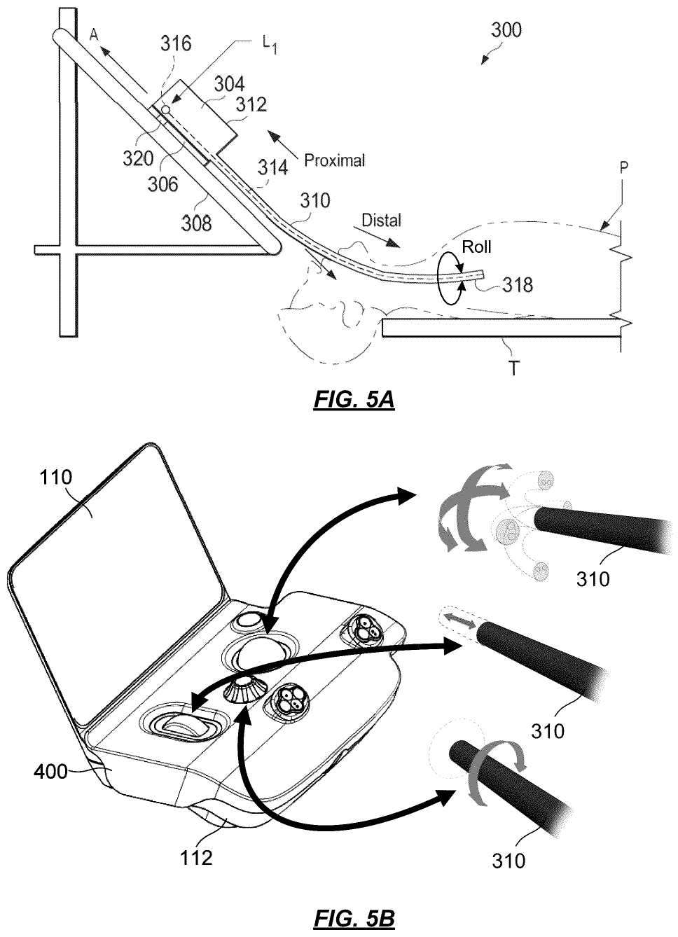

FIG. 5A is a simplified diagram of a side view of a patient coordinate space including a medical instrument 304 with an additional degree of freedom according to some embodiments.

In FIG. 5A, the instrument body 312 and the instrument carriage 306 are shown in an advanced position along the insertion stage 308. In other words, relative to FIG. 3A, the instrument body 312 and the instrument carriage 306 have advanced along the linear track of insertion stage 308. Accordingly, the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

As discussed above, the surgical instrument control device 112 controls insertion motion (e.g., motion along an insertion axis A) and/or motion of the distal end 318 of the elongate device 310 in yaw/pitch/roll direction(s). In one or more embodiments, the distal end 318 of the elongate device 310 may be configured to move in a roll direction (e.g., along the longitudinal axis of the distal end 318). Because the surgical instrument control device 112 does not include a control element for the roll DOF by default, a control shell 400 may be installed on the surgical instrument control device 112 to provide control over the additional DOF.

FIG. 5B is a simplified perspective diagram of the surgical instrument control device 112 in FIG. 3B and a control shell 400 according to some embodiments.

In some embodiments, shell control element 432 is a single degree of freedom infinite length of travel input control providing infinite length of travel about a roll axis usable by the operator to control the roll position of the distal end 318 of the elongate device 310. Shell control element 432 is depicted as a scroll knob, however, other types of input controls (e.g., scrolling an annual ring, scrolling a wheel, twisting a trackball, detecting a relative or absolute touch location on a trackball/knob/wheel/ring), including non-infinite length of travel input controls, are possible. In some examples, scrolling of the knob clockwise increases the roll position in a clockwise direction about a longitudinal vector protruding from the distal end 318 of the elongate device 310 and scrolling of the knob in a counterclockwise direction decreases the roll position. In some examples, the roll direction is reversed (e.g., with respect to a longitudinal vector entering the distal end 318 of the elongate device 310).

When shell control element 432 is an infinite length of travel input control, operating shell control element 432 in a position-specifying mode allows the operator to exercise precise roll control of the distal end 318 of the elongate device 310. In some examples, movement of shell control element 432 may be detected by the one or more circuit boards, logic boards, and/or the like of control shell 400 using one or more encoders, resolvers, optical sensors, hall effect sensors, and/or the like (not shown). In some examples, feedback applied via one or more electromagnetic actuators, and/or the like may optionally be used to apply haptic feedback to shell control element 432. In some examples, a scale factor between an amount of movement of shell control element 432 and an amount of roll movement by the elongate device 310 is adjustable by the operator and/or control software so that a roll velocity relative to an angular velocity of shell control element 432 may be adjusted to allow both fast roll motion when advantageous and slower more precise roll motion when greater control precision is desired.

In some embodiments, shell control element 432 may include a touch sensor. The touch sensor may be a capacitive touch sensor, any other type of touch sensor (e.g., a luminosity sensor that detects reflected or scattered light, a contact sensor, a distance sensor), or any combination of sensors. Alternatively or additionally, a pressure sensor may be included. The touch and/or pressure sensor may be used to differentiate intended movement by the operator from inadvertent movement due to accidental contact (e.g., lock a function of the control shell 400 or the surgical instrument control device 112). Other types of proximity sensors (e.g., ultrasonic sensors, vision sensors, light walls, and/or the like) may be used to detect operator proximity to the shell control element 432. In some examples, one or more wrist detection sensors (e.g., capacitive touch, pressure, and/or similar sensors) in a wrist rest may be used to detect operator proximity to the shell control element 432.

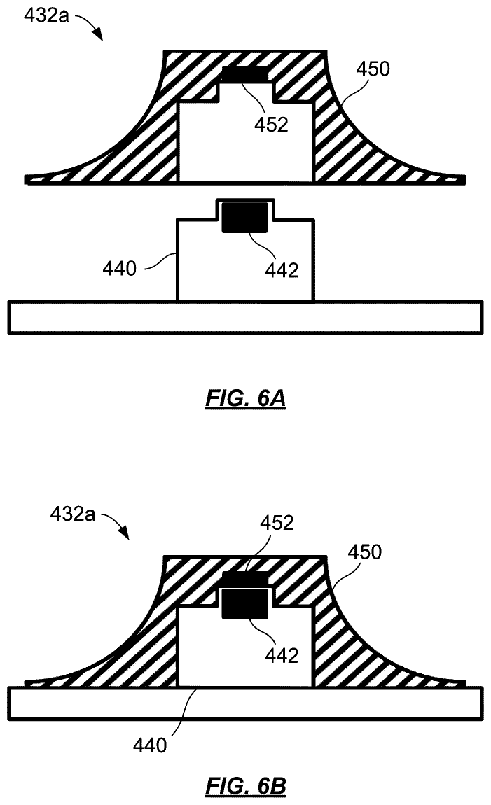

As described in further detail below with respect to FIGS. 6A-6B, in some embodiments, the shell control element 432 may include additional features beyond providing an input for motion control or action control.

FIG. 6A is a simplified diagram of a side view of a shell control element 432a in a first configuration according to some embodiments.

In some embodiments, the shell control element 432a may function as a key that locks/unlocks a function of the control shell 400 and/or surgical instrument control device 112. The shell control element 432a may include two separate parts: a lock component 440 disposed on the body 402 of the control shell 400; and a key component 450 that is removable from the lock component 440. When the key component 450 is removed from the lock component 440, as shown in FIG. 6A, the control shell 400 may be configured to disable or lock a function of the control shell 400 (e.g., prevent input from the shell control element 432a or any/all other shell control element(s)) and/or the surgical instrument control device 112 (e.g., prevent input from one or more preexisting control elements). In other words, based on a hardware configuration of the shell control element 432a, the control shell 400 may secure the manipulator assembly 102 from unintended or unauthorized use.

In one or more embodiments, the shell control element 432a includes a removable cover as the key component 450 and a base portion as the lock component 440. In some embodiments, the key component 450 includes a first magnet 452 and the lock component 440 includes a second magnet 442. The first and second magnets 442, 452 may secure the removable cover to the base portion, as shown in FIG. 6B. In some embodiments, the removable cover and the base portion include a notch and a corresponding groove that require the key component 450 and lock component 440 to be oriented in specific orientation to connect.

FIG. 6B is a simplified diagram of a side view of the shell control element of FIG. 6A in a second configuration according to some embodiments.

In some embodiments, the lock component 440 includes an embedded detector that detects whether the key component 450 has been removed from the lock component 440. For example, the second magnet 442 may include an electromagnetic sensor may be used to detect the proximity of the first magnet 452 and/or a whether the first and second magnet 442, 452 are connected to determine whether the key component 450 has been removed/attached from the lock component 440. Other detectors may be used (e.g., proximity sensors, ultrasonic sensors, optical sensors, capacitive touch, pressure, and/or similar sensors) to detect the key component 450.

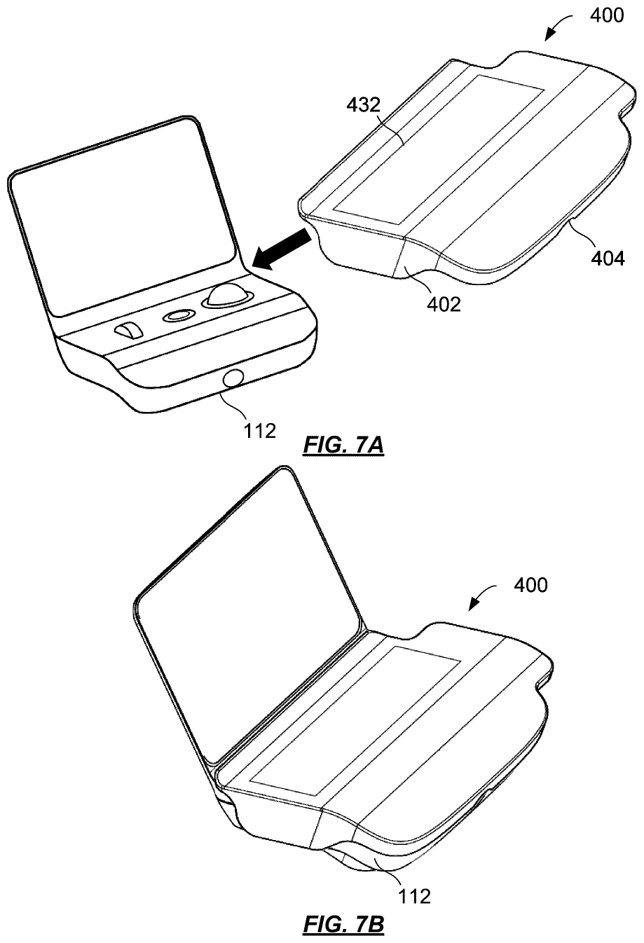

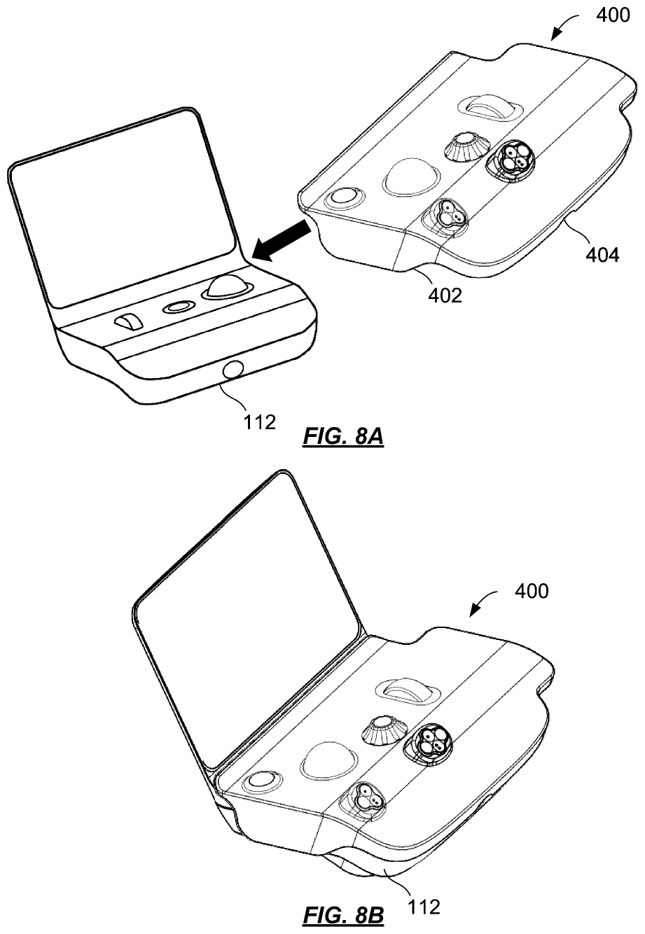

FIGS. 7A-7B are simplified perspective diagrams of a control shell 400 and a surgical instrument control device 112 according to some embodiments.

In some embodiments, the control shell 400 is configured to replace the control scheme of the surgical instrument control device 112. For example, the control shell 400 may include a shell control element 432 comprising a touch interface. The touch interface may be a tablet device that presents the operator with virtual controls for the manipulator assembly 102.

In some embodiments, the touch interface may replace functionality of one or more preexisting control elements of the surgical instrument control device 112. As shown in FIG. 7A, the preexisting control elements (i.e., the insertion/retraction control 232a, the passive control button 232b, the steering control 232c) of the surgical instrument control device 112 are obscured from the operator by the body 402 of the control shell 400. In some embodiments, the control shell 400 is configured to disable a preexisting control element that is replaced by the functionality of the shell control element 432 (e.g., based on the instructions stored in the memory, the processor is configured to disable the preexisting control element).

The control shell 400 may be configured to retain functionality of one or more preexisting control elements. For example, the body 402 may still include a cutout region 404 allowing at least one preexisting control element (e.g., the emergency stop button 232d) to remain accessible through the control shell to the operator.

FIGS. 8A-8B are simplified perspective diagrams of a control shell 400 and a surgical instrument control device 112 according to some embodiments.

In some embodiments, the control shell 400 is configured to rearrange the control scheme of the surgical instrument control device 112. For example, the control shell 400 may include a plurality of shell control elements 432 in a layout that mirrors, relative to a traverse axis of the surgical instrument control device 112, a layout of preexisting control elements of a first handedness on the surgical instrument control device 112. As shown in FIG. 8A, the surgical instrument control device 112 may be considered to have a first handedness with the insertion/retraction control 232a on a left transverse side and the steering control 232c on a right transverse side.

In contrast, the corresponding shell control elements 432 are mirrored with the insertion/retraction control on the right transverse side and the steering control on the left transverse side. In other words, the layout of the shell control elements 432 have a second handedness that is opposite to the first handedness of the surgical instrument control device 112. In some embodiments, the body 402 of the control shell 400 obscures the preexisting control elements of the first handedness on the surgical instrument control device 112 such that they are not physically accessible to the operator.

In some embodiments, the control shell 400 mirrors the layout of preexisting control elements without obscuring the preexisting control elements. Instead, the control shell 400 may disable the preexisting control elements of the first handedness (e.g., based on the instructions stored in the memory, the processor is configured to disable the preexisting control element).

The control shell 400 may be configured to retain functionality of one or more preexisting control elements of the surgical instrument control device 112. For example, the body 402 may still include cutout regions 404 for the passive button 232b and the emergency stop button 232d to remain accessible through the control shell to the operator.

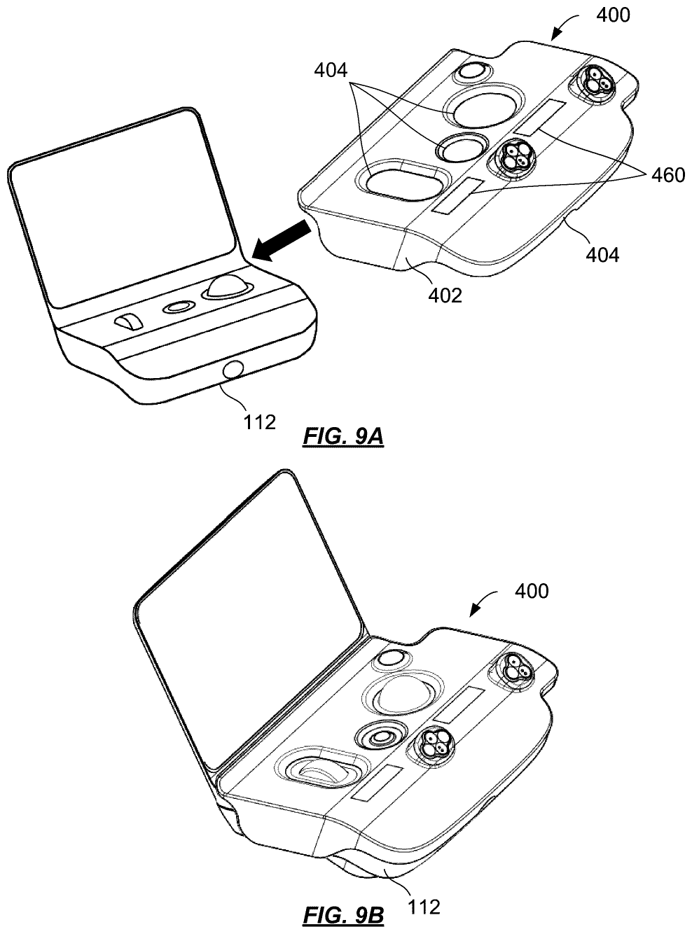

FIGS. 9A-9B are simplified perspective diagrams of a control shell 400 and a surgical instrument control device 112 according to some embodiments.

In some embodiments, the control shell 400 is configured to visually augment the control scheme of the surgical instrument control device 112. For example, the control shell 400 may include a user feedback device (e.g., a lighting system) in additional to a shell control element (not shown). For example, as a user feedback device, a lighting system may include a plurality of lighting elements 460 that correspond to preexisting control elements of the surgical instrument control device 112. The control shell 400 may guide an operator through a procedure by activating the lighting system based on procedure-specific instructions.

The control shell 400 may be configured to retain functionality of all preexisting control elements of the surgical instrument control device 112. For example, the body 402 may still include cutout regions 404 for some or all preexisting control elements to remain accessible through the control shell 400 to the operator.

In some embodiments, the control shell 400 may augment the functionality of a preexisting control element. The control shell 400 may provide the surgical instrument control device 112 with new control information for a preexisting control element 232 to create a new control scheme for the preexisting control element 232. For example, a track ball configured to control yaw and pitch movement (e.g., corresponding to x-axis scrolling and y-axis scrolling of the track ball, respectively) may be augmented to further control a roll movement based on the new control information (e.g., corresponding to a spinning about a z-axis of the track ball).

In some embodiments, the control shell 400 may include control and/or information display capabilities for a new instrument of the teleoperated surgical system. For example, the preexisting control scheme of the surgical instrument control device 112 may include instructions for controlling an operation (e.g., a first robotic movement) of a first instrument (e.g., a first endoscope without roll functionality) according to a first user input received at the surgical instrument control device. To account for a new instrument, the control shell 400 may provide the surgical instrument control device 112 with new control information including instructions for controlling (e.g., moving (e.g., executing a robotic movement), operating, managing) the new instrument according to a user input received at the control shell (e.g., via one or more shell control elements). For example, the control information may include instructions for controlling a second robotic movement (e.g., a roll movement) of the new instrument (e.g., a second endoscope with roll functionality) according to a second user input received at the shell control element. In some embodiments, the second instrument is configured to perform the first robotic movement (e.g., the second endoscope with roll functionality can perform all movements of the first endoscope without roll functionality) but the first instrument is not configured to perform the second robotic movement (e.g., the first endoscope without the roll functionality cannot perform the roll movement of the second endoscope with the roll functionality). Accordingly, while the surgical instrument control device is not original configured to receive the inputs for operating the new instrument, the control shell 400 provides the physical inputs (i.e., shell control elements 432) and control information for receiving inputs for operating the new instrument.

In other words, the control shell provides a new control scheme for a teleoperated surgical system that has been augmented with a new instrument and/or functionality.

While various embodiments of the control shell 400 and shell control elements 432 have been described with respect to FIGS. 6A-9B, it will be appreciated that other embodiments may be conceived without deviating from the gist of the disclosure. For example, different arrangements of shell control elements 432 and/or cut out regions 404 may be combined to create a control scheme specific to the teleoperated surgical system, the manipulator assembly, and/or any additional equipment of the teleoperated surgical system (e.g., connected to the manipulator assembly).

The control shell 400 may be configured for any customization (e.g., operator-specific layout). For example, different control shells 400 may be configured for different operators of the medical system 100, based on their preferences (e.g., control sensitivity, handedness, etc.). Accordingly, the surgical instrument control device 112 may be reconfigured by removing and installing different control shells 400 for different operators.

In some embodiments, each of a plurality control shells 400 may be configured for a specific procedure utilizing the medical system 100. The control shell 400 may be configured to lock and unlock a function of the surgical instrument control device 112 and/or the shell control element 432 based on procedure-specific instructions. Accordingly, the surgical instrument control device 112 may be reconfigured by removing and installing different control shells 400 for different procedures.

In a non-limiting example, the control shell 400 may be configured for an endoscopic procedure. An endoscopic instrument may inserted through a cannula of an endoscope or otherwise attached an endoscope to perform the endoscopic procedure (e.g., a cutting tool to perform dissection or resection, a suturing tool to stitch together anatomy).

Accordingly, a shell control element 432 may include an input device for manipulating an endoscopic instrument of the medical system 100. For example, the shell control element 432 may trigger plicating or suturing actions to stitch together folds of a stomach to reduce its size (e.g., endoscopic sleeve gastroplasty) or reduce the size of gastrointestinal inlets/outlets (e.g., transoral outlet reduction).

In another non-limiting example, the control shell 400 may be configured for a navigation procedure. Accordingly, a shell control element 432 may be configured to control a movement DOF of the manipulator assembly 102. A navigation procedure may be a part of a diagnostic procedure (e.g., esophagogastroduodenoscopy (EGD), where you navigate and visualize the anatomy), a non-diagnostic procedure (e.g., a surgical procedure), or any procedure that requires navigation of the manipulator assembly 102.

In another non-limiting example, the control shell 400 may be configured for a visualization procedure. Accordingly, a shell control element 432 may be configured to control an imaging sensor connected to the manipulator assembly 102. A visualization procedure may be a part of a diagnostic procedure (e.g., EGD, where you navigate and visualize the anatomy), a non-diagnostic procedure (e.g., a surgical procedure), or any procedure that utilizes an imaging sensor connected to the manipulator assembly 102.

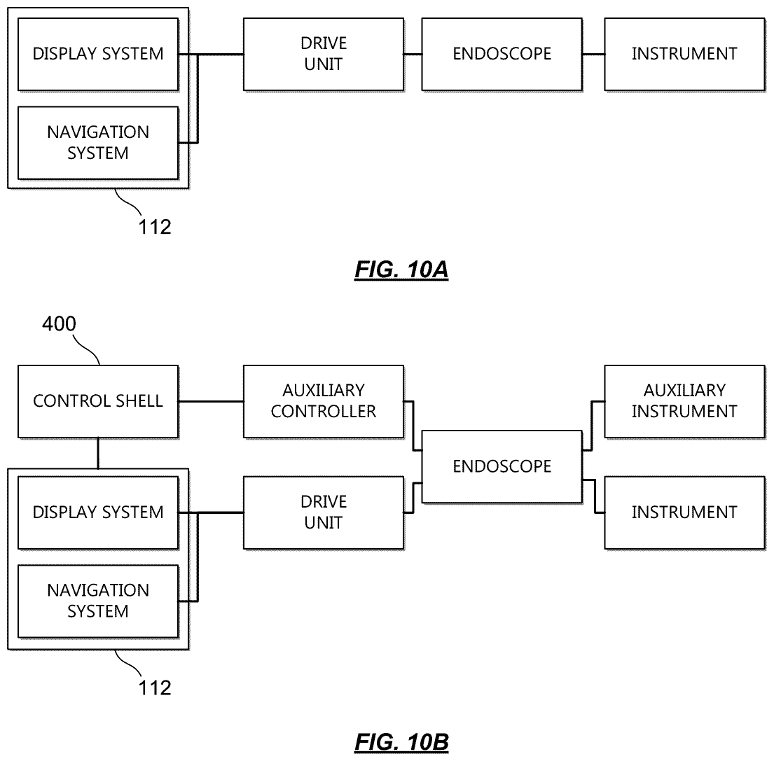

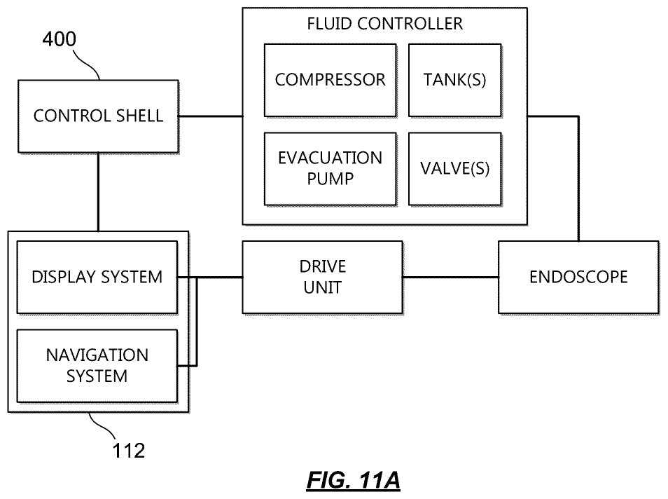

In another non-limiting example, the control shell 400 may be configured for operating a system that is separate from the manipulator assembly 102 and that operates in coordination with the manipulator assembly 102. This configuration is described in further detail below with respect to FIGS. 10A-11B.

FIG. 10A is a simplified schematic of a medical system.

As discussed above with respect to FIGS. 1-2A, a medical system 100 may include a manipulator assembly 102 comprising an endoscope that supports a medical instrument 104. The surgical instrument control device 112 include a navigation system 223 and a display system 110. By operating the surgical instrument control device 112, an operator may control the endoscope and medical instrument 104 (e.g., advance medical instrument 104 into a naturally or surgically created anatomic orifice). The medical instrument 104 may include an imaging tool and/or a surgical tool to perform a procedure (e.g., suturing, dissection, resection, biopsy, etc.).

FIG. 10B is a simplified schematic of the medical system of FIG. 10A with a separate system according to some embodiments.

While the medical system of FIG. 10A may be used to perform a variety of procedures using the medical instrument 104, an operator may expand the range of available procedures by adding an auxiliary instrument to the medical system. The auxiliary instrument may be part of a separate system that requires an additional control input that is not included in the surgical instrument control device 112 (e.g., a third-party system, an after-market modification). As shown in FIG. 10B, the control shell 400 may be used as an interface between the auxiliary controller of the separate system and the surgical instrument control device 112. In some embodiments, the control shell 400 includes cutout regions 404 to keep the preexisting control elements accessible to the operator while providing one or more new control elements to control the functionality of the auxiliary instrument. Accordingly, the operator may use the endoscope and medical instrument 104 with the additional capabilities of the separate system.

FIG. 11A is a simplified schematic of a medical system with an auxiliary fluid control system according to some embodiments.

In some embodiments, the control shell 400 is configured to control an fluid system connected to the manipulator assembly 102. The fluid system may include one or more subsystems for inserting or removing a fluid (e.g., liquid, gas, suspension of solids, or any appropriate material) from a distal end of the endoscope. For example, the fluid system may supply a fluid to the region around the distal end of the endoscope (e.g., insufflation, irrigation, washing) or remove a fluid from region around the distal end of the endoscope (e.g., suction, evacuation, tissue removal, smoke removal).

As shown in FIG. 11A, the fluid system includes a fluid controller comprising one or more of the following: a compressor to provide positive pressure; a vacuum pump to provide “negative” pressure; a tank to store a fluid; and a valve to control flow of a fluid. The fluid system further includes fluid conduits/ports/manifolds (not shown) and any necessary infrastructure to manage the fluid movement through the manipulator assembly 102.

FIG. 11B is a simplified perspective diagram of a control shell 400 installed on a surgical instrument control device 112 for the medical system of FIG. 11A according to some embodiments.

The control shell 400 of FIG. 11B includes a plurality of shell control elements 432 to operate the fluid system of FIG. 11A. The control shell 400 includes cutout portions disposed to allowed the insertion/retraction control 232a, the steering control 232c, and the emergency stop button 232d of the surgical instrument control device 112 to protrude through the control shell 400 and remain accessible to the operator. While the passive control button 232b is obscured by the control shell 400, a button shell control element (e.g., the button closest to the display system 110) may be included on the control shell 400 to provide an alternate method of activating the functions associated with the passive control button 232b.

Furthermore, the roll knob configured to control the roll position of the endoscope is disposed in a central region of the control shell 400 (e.g., between the scroll wheel on the left and the trackball on the right) to provide access from either hand (e.g., ambidextrous operation). In addition, the central location may be a critical requirement for procedures that utilize simultaneous insertion insertion/roll control (i.e., left scroll wheel and central roll knob) and simultaneous flex/roll control (i.e., central roll knob and right trackball). Furthermore, in some embodiments, the height of the roll knob relative to the control shell 400 may match the height of one or more of the other control elements (e.g., left scroll wheel, right trackball) relative to the control shell 400 (e.g., avoid unintentional activation, promote ease of simultaneous control).

Similar to the central roll knob, a collection of one or more button control elements may be disposed in a central region of the control shell 400 to provide access to a primary set of fluid controls from either hand. For example, as shown in FIG. 11B, the control shell 400 includes passive button control elements for different series of actions configured to be performed by the fluid system: insufflation (e.g., open a valve at a fluid tank, activate a compressor for a predetermined amount of time, emission of a fluid from the elongate device into the insertion region, close the valve at the fluid tank); suction (e.g., activate a vacuum pump for a predetermined amount of time, open a valve to access the insertion region, evacuation of a fluid from the insertion region by the elongate device; close the valve); lens wash/clean (e.g., activate a lens washer installed at the distal end of the endoscope). An additional collection of button control elements (e.g., one or more duplicate control elements and/or additional control elements) may be disposed along an edge of the control shell to provide a secondary method of accessing the fluid controls.

A collection of multiple button control elements may be arranged in any configuration. For example, the button control elements may be arranged in a diamond pattern or in a linear vertical “stop-light” pattern. One or more of the button control elements may include a reference mark (e.g., a pattern of protrusions or divots) to provide the operator with a tactile method of identifying the one or more buttons within the configuration (e.g., a topmost button, a lowermost button).

In some embodiments, the collection of buttons may be offset from a surface of the control shell 400 for ergonomic purposes. For example, a surface that support the collection of buttons may be oriented at an angle (e.g., 10, 12, 15, 20 degrees) relative to a palm rest portion of the control shell 400 to provide ease of access. The angle may be determined based on neutral hand positioning of the operator (e.g., a specific operator, a population of operators).

In some embodiments, the control shell 400 includes a shell control element to control an irrigation subsystem (e.g., an irrigation pump for fluid emission and/or fluid suction) of the fluid control system. The shell control element may be a foot pedal connected to the control shell 400 by a wired or wireless connection interface 412.

The layout of the control shell 400 shown in FIG. 11B may streamline the workflow while an endoscope is located to the right of the user. Accordingly, the control shell 400 of FIG. 11B may be mirrored, relative to a traverse axis of the surgical instrument control device 112, to streamline the workflow while the endoscope is located to the left of the user.

While FIG. 11B describes a fluid system as a system that is separate from the manipulator assembly and that operates in coordination with the manipulator assembly, it will be appreciated that other embodiments may be conceived without deviating from the gist of the disclosure. In general, the control shell 400 may be configured as a physical interface for any system or subsystem of the teleoperated surgical system. Accordingly, the shell control element of the control shell 400 may control a mode or an operation of a manipulator assembly and/or a separate system from the manipulator assembly. For example, a fluid system may be configured with greater or fewer elements/features than describe above, requiring a different configuration of the control shell 400. In some embodiments, the separate system may be an energy system and a shell control element of the control shell 400 is configured to control a mode or an operation of the energy system. In some embodiments, the separate system may be an imaging system and a shell control element of the control shell 400 is configured to control a mode or an operation of the imaging system.

FIGS. 11C-11D are simplified perspective diagrams of an alternate control shell 400 installed on an surgical instrument control device 112 for the medical instrument system of FIG. 11A according to some embodiments.

In some embodiments, the control shell 400 is configured with cutout regions 404 corresponding to a scroll wheel 232a (e.g., controls insertion of the endoscope of the medical system) and a track ball 232c (e.g., controls yaw and pitch movement of the endoscope) of the surgical instrument control device 112 (i.e., the preexisting input device, the surgical instrument control device). To control a single new DOF of the manipulator assembly 102 that is not controlled by the surgical instrument control device 112 (e.g., a roll DOF added to the endoscope), the control shell 400 may include an annular scrolling wheel 432a (e.g., infinite length of travel, non-infinite length of travel input control).

FIG. 11E is a simplified perspective diagrams of an external portion of the alternate control shell 400 of FIG. 11C according to some embodiments.

In some embodiments, the annular scrolling wheel 432a surrounds the cutout region 404 corresponding to the track ball 232c of the surgical instrument control device 112. Accordingly, the combination of the surgical instrument control device 112 and the control shell 400 provide the operator with controls for pitch, yaw, and roll of the endoscope at a single location that is accessible to both left-and right-handed operators. Furthermore, the shell control element 432b may include two collections of multiple buttons disposed on opposite sides of the annular scrolling wheel 432a to provide ambidextrous control access for additional functions of the medical system.

FIG. 11F is a simplified perspective diagrams of an internal portion of the alternate control shell 400 of FIG. 11C according to some embodiments.

In some embodiments, the annular scrolling wheel 432a includes a toothed gear 432d read by an encoder. The encoder may include a resolver, an optical sensor, a hall effect sensor, and/or the like. In some examples, the encoder directly reads the rotation of the annular scrolling wheel 432a. In some examples, the encoder includes a toothed gear 432f that modifies (e.g., scales by a tooth ratio) the rotation of the annular scrolling wheel 432a.

In some embodiments, the annular scrolling wheel 432a may be locked (e.g., a hardware lock to prevent rotation, a software lock to ignore input from the encoder) to prevent unintended motion. For example, a touch sensor (e.g., included in the annular scrolling wheel 432a, included in another shell control element, embedded in the body 402 of the control shell 400) may detect the presence or absence of the operators hand and enable or disable the lock accordingly.

FIG. 12 is a simplified perspective diagram of an alternate control shell installed on an input control console for the medical instrument system of FIG. 11A according to some embodiments.

Similar to FIG. 11B, the control shell 400 of FIG. 12 includes a plurality of shell control elements 432 to operate the fluid system of FIG. 11A. However, the control shell 400 of FIG. 11C includes cutout portions disposed to allowed the insertion/retraction control 232a, the passive control button 232b, the steering control 232c, and the emergency stop button 232d of the surgical instrument control device 112 to protrude through the control shell 400 and remain accessible to the operator.

In some embodiments, the control shell includes one or more passive button control elements corresponding to different functions of the fluid system. For example, a first button control element may control emitting one or more fluids from the manipulator assembly (e.g., a water jet), a second button control element may control evacuation of one or more fluids by the manipulator assembly (e.g., suction), and a third button control element may control a tool connected to the distal end of the manipulator assembly 102 (e.g. a lens washer installed at the distal end of the endoscope). Operating the each of the button control elements may cause the fluid system to perform a series of actions (e.g., open a valve at a fluid tank, activate a compressor for a predetermined amount of time, close the valve at the fluid tank).

In some embodiments, the control shell 400 includes a foot pedal control element to control another function of the fluid system. For example, the foot pedal may control an irrigation or insufflation subsystem connected to the manipulator assembly 102. The foot pedal control element may be connected to the control shell 400 by a wired or wireless connection interface.

In some embodiments, one or more shell control elements 432 may control a tool connected to the manipulator assembly 102. For example, a button may be used to activate a lens washer connected to a distal end of an endoscope. Operating the shell control element 432 may execute a series of actions in the fluid system (e.g., open a valve at a fluid tank, activate a compressor for a predetermined amount of time, close the valve at the fluid tank). In another example, the shell control element 432 may control a tool (e.g., a suturing tool, a manipulator, a camera) inserted through a cannula of the endoscope.

FIG. 13 is a schematic diagram that shows an example of a computing system 1300, in accordance with one or more embodiments.

The computing system 1300 can be used for some or all of the operations described previously, according to some implementations. The computing system 1300 may include a processor 1310, a memory 1320, a storage device 1330, and an input/output device.

Each of the processor 1310, the memory 1320, the storage device 1330, and the input/output device are interconnected using a system bus 1340. The processor 1310 is capable of processing instructions for execution within the computing system 1300. In some implementations, the processor 1310 is a single-threaded processor. In some implementations, the processor 1310 is a multi-threaded processor. The processor 1310 is capable of processing instructions stored in the memory 1320 or on the storage device 1330 to display graphical information for a user interface on the input/output device.

The memory 1320 stores information within the computing system 1300. In some implementations, the memory 1320 is a computer-readable medium. In some implementations, the memory 1320 is a volatile memory unit. In some implementations, the memory 1320 is a non-volatile memory unit.

The storage device 1330 is capable of providing mass storage for the computing system 1300. In some implementations, the storage device 1330 is a computer-readable medium. In various different implementations, the storage device 1330 may be a floppy disk device, a hard disk device, an optical disk device, or a tape device.

The input/output device provides input/output operations for the computing system 1300. In some implementations, the input/output device includes a keyboard and/or pointing device. In some implementations, the input/output device includes a display unit for displaying graphical user interfaces. In some implementations, the input/output device includes control elements of the surgical instrument control device 112 and/or the control shell 400.

Some features described can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic disks such as internal hard disks and removable disks; magneto-optical disks; and CD-ROM (compact disc read-only memory) and DVD-ROM (digital versatile disc read-only memory) disks. The processor and the memory can be supplemented by, or incorporated in, ASICs (application-specific integrated circuits).

To provide for interaction with an operator, some features can be implemented on a computer having a display device such as a CRT (cathode ray tube) or LCD (liquid crystal display) monitor for displaying information to the operator and a keyboard and a pointing device such as a mouse or a trackball by which the operator can provide input to the computer.

Some features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN (local area network), a WAN (wide area network), and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

FIG. 14 is a flowchart of a method 1400 of operating a teleoperated surgical system according to some embodiments.

In FIG. 14, the flowchart shows the method 1400 for operating a teleoperated surgical system including a control shell that integrates with a surgical instrument control device of a teleoperated surgical system.

Integrating the control shell with the surgical instrument control device includes communicatively connecting the control shell to the surgical instrument control device. In some embodiments, a communication interface of the control shell is configured to directly connect the control shell to the surgical instrument control device. In some embodiments, a communication interface of the control shell may be configured to directly connect the control shell to a second system that is separate from the manipulator assembly such that information is transferrable from the control shell to the surgical instrument control device via the second system. In other words, communications may be routed between the control shell, the surgical instrument control device, the second system, or any system/sub-system of the teleoperated surgical system based on any suitable communication protocol.

At 1405, the control shell transmits, to the surgical instrument control device, control information corresponding to a shell control element of the control shell. Control information (e.g., configuration information, calibration information) allows the control shell to communicate properties and attributes to the surgical instrument control device. For example, the control information may include a correlation between a function of the teleoperated surgical system and the shell control element.

For example, the control information may include calibration information (e.g., a correlation between actuation of the shell control element and a movement of a manipulator assembly) for the shell control element. While various non-limiting embodiments of calibration information are described below with respect to different types of shell control elements, it will be appreciated that other embodiments may be conceived without deviating from the gist of the disclosure.

In the case of the shell control element having a single degree of freedom (e.g., a scrolling knob/dial/annular wheel) that corresponds to a movement degree of freedom of the manipulator assembly, the calibration information may include a scaling factor that converts an amount of actuation of the shell control element to an amount of movement in the manipulator assembly. For example, in the case of the shell control element that controls a roll motion of an endoscope of the manipulator assembly, the calibration information may include a scaling factor that converts actuation of the shell control element to an amount of rotation about a roll axis of the endoscope.

In some embodiments, the shell control element is a scroll wheel with a single degree of freedom that controls insertion and retraction of an endoscope of the manipulator assembly. Therefore, the calibration information may include a scaling factor that converts a rotation amount of the scroll wheel to an amount of insertion or retraction of the endoscope.

In the case of the shell control element having a multiple degrees of freedom that correspond to different degrees of freedom of the manipulator assembly, the calibration information may include multiple scaling factors that converts an amount of actuation in each of the different degrees of freedom of the shell control element to an amount of movement in the corresponding degrees of freedom of the manipulator assembly.

For example, in the case of the shell control element that is a track ball with multiple rotational degrees of freedom (e.g., x-rotation/roll, y-rotation/roll, and/or z-rotation/roll), the calibration information may include multiple scaling factors that convert a rotation amount in each of the different rotational degrees of freedom of the track ball to an amount of actuation in the corresponding degrees of freedom of the manipulator assembly. In some embodiments, x-rotation/roll and y-rotation/roll of the track ball may control pitch rotation and yaw rotation of an endoscope or portion of the endoscope (e.g., flexing of a distal portion of the endoscope) of the manipulator assembly. Accordingly, the calibration information may include scaling factors that convert x/y rotation amounts of the track ball to an amount of pitch rotation and yaw rotation of the endoscope, respectively. In some embodiments, z-rotation/roll of the track ball may control a roll motion of an endoscope of the manipulator assembly.

Therefore, the calibration information may further include a scaling factor that converts z-rotation of the track ball to an amount of rotation about a roll axis of the endoscope.

In the case of the shell control element that is a togglable input, the calibration information may include a routing table that associates the togglable input with a function of the manipulator assembly or a system (e.g., a second system) that is separate from the manipulator assembly and that operates in coordination with the manipulator assembly (e.g., a fluid system as shown in FIG. 11A). For example, actuating a button or a togglable switch on the control shell may be linked with activating or deactivating a function or action to be executed by the manipulator assembly or the second system. In the case of a fluid system as the second system, a button or a togglable switch may be associated with fluid emission or fluid evacuation function. In the case of a plurality of shell control elements, a routing table may be used to associate the plurality of shell control elements with functions of the manipulator assembly or the second system.

In the case of the control shell including a user feedback device, the calibration information may include instructions (e.g., rules for activation/deactivation) for operative the user feedback device. For example, in the case of the shell control element that is a lighting element, the calibration information may include instructions (e.g., rules for activation, color change, and/or user interface) for illuminating the lighting element. In some embodiments, the calibration information may modify a control scheme stored in the surgical instrument control device to illuminate the lighting element based on use of a preexisting control element (e.g., illuminating the lighting element indicates the preexisting control element is enabled). For example, the calibration information may be based on a specific procedure that utilizes the preexisting control element (e.g., illuminating the lighting element indicates the preexisting control element is the next input in a procedure). In some embodiments, the calibration information may modify a control scheme stored in the surgical instrument control device to illuminate the lighting element based on use of a shell control element (e.g., illuminating the lighting element indicates the shell control element is enabled and/or is the next input in a procedure).

In the case of a second system that is separate from the manipulator assembly and that operates in coordination with the manipulator assembly, the control information transmitted at 1405 includes instructions for modifying of the preexisting control scheme to create a new control scheme. For example, the new control scheme may include mappings and/or associations for each of the control elements on the surgical instrument control device and the control shell. In some embodiments, the new control scheme may include: a first mapping of control elements of the surgical instrument control device to functions of the manipulator assembly; and a second mapping of the shell control element of the control shell to a function of the second system.

In some embodiments, the control information transmitted from the control shell to the surgical instrument control device at 1405 includes instructions for when the control shell is removed or disconnected from the surgical instrument control device. For example, the control information may instruct the surgical instrument control device to store the previous control scheme in memory for later use (e.g., reinstating a previous control scheme when the control shell is removed or disconnected from the surgical instrument control device). The control information may include instructions for removing or disconnecting the control shell during operation of the teleoperated surgical system (e.g., hot-swapping control schemes). In some cases, the control information may include instructions for handling unexpected disconnection of the control shell (e.g., revert to previous control scheme, emergency stop actions and/or instructions).

At 1410, a preexisting control scheme stored in the surgical instrument control device is modified to include the control information.

In some embodiments, modifying of the preexisting control scheme may include remapping a preexisting control element of the surgical instrument control device. While various non-limiting embodiments of remapping a preexisting control element are described below, it will be appreciated that other embodiments may be conceived without deviating from the gist of the disclosure.

Remapping a preexisting control elements may include changing calibration information for the preexisting control element. For example, the calibration information include a change to a sensitivity or a threshold parameter for the preexisting control element.

Remapping of a preexisting control elements may include disabling the preexisting control element. For example, when the control shell is designed with a specific procedure in mind, the disabled preexisting control element may correspond to a function that is prohibited during the specific procedure (e.g., preventing accidental activation of the prohibited function). In some embodiments, the function of the disabled preexisting control element may be remapped to the control shell (e.g., transferring a function of the surgical instrument control device to the control shell, repositioning the function of the surgical instrument control device to a different position to improve ergonomics (e.g., based on the control scheme for the specific procedure)).

At 1415, initializing the teleoperated surgical system to receive and process both the second user input from the control shell element and the first user input from the surgical instrument control device. The teleoperated surgical system may be operated from the control shell in combination with the surgical instrument control device. The combination of shell control element(s) and the control element(s) of the surgical instrument control device cumulatively allow a user to operate the teleoperated surgical system (e.g., a manipulator assembly, a second system that is separate from the manipulator assembly and that operates in coordination with the manipulator assembly, and/or any system or subsystem of the teleoperated surgical system).

Optionally, at 1420, the control shell transmits an input or an instruction corresponding to the input in response to the input at the shell control element. The input may be transmitted to the surgical instrument control device (e.g., transmitted directly from the control shell to the preexisting control, transmitted indirectly from the control shell to the preexisting control via a second system). The input may be transmitted to a second system that is separate from the manipulator assembly and that operates in coordination with the manipulator assembly (e.g., transmitted directly from the control shell to the second system).

In the case of the control shell having a plurality of shell control elements, in some embodiments, the control shell aggregates a combination of inputs (or instructions corresponding to the inputs) from the plurality of shell control elements during a time window.

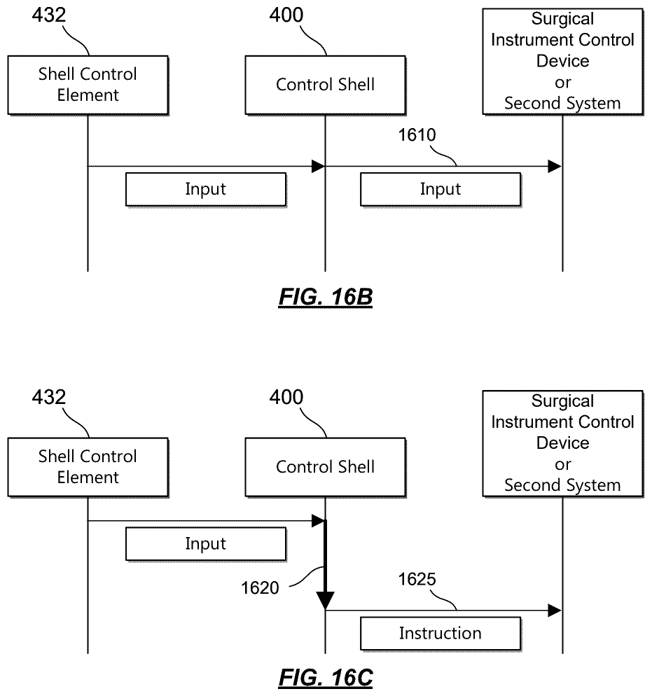

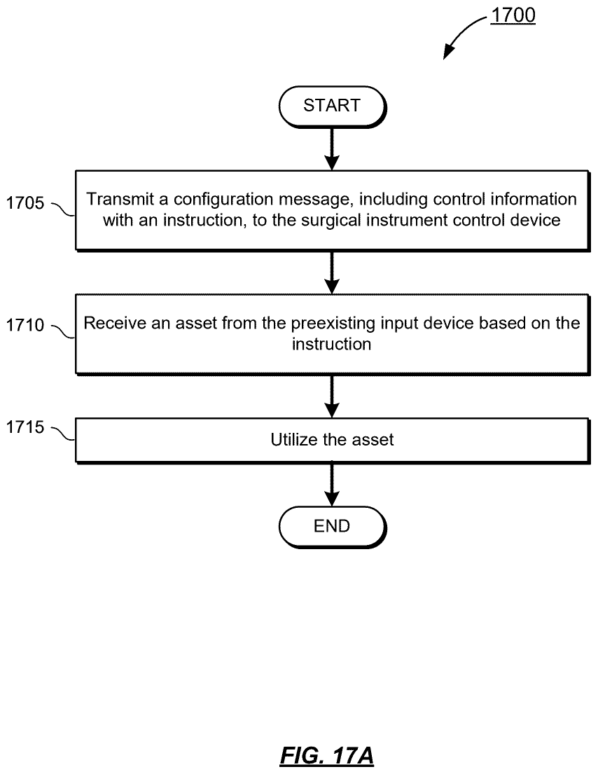

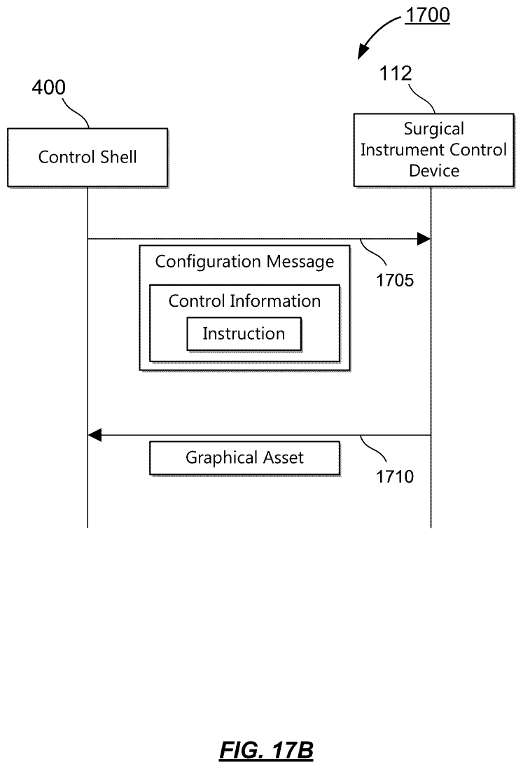

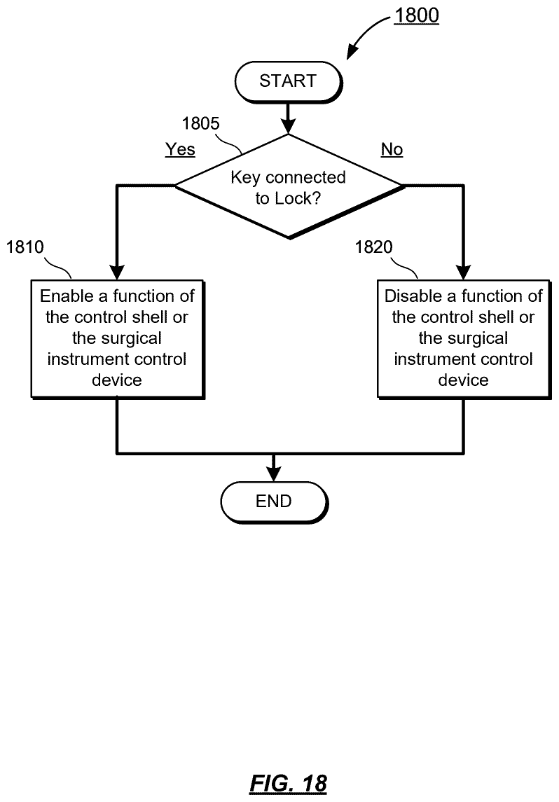

The determination to transmit the input itself or an instruction corresponding to the input may be based on a type of communication mode established between the control shell and the surgical instrument control device, as described in further detail below with respect to FIGS. 16A-16C.

Optionally, at 1425, the teleoperated surgical system is operated based on the input or the instruction transmitted from the control shell. For example, a function of the manipulator assembly (e.g., movement, actuation) and/or a function of a second system that is separate from the manipulator assembly and that operates in coordination with the manipulator assembly is executed based on the input or instruction.

In some embodiments, when the control shell is no longer required, the control shell may be removed or disconnected from the surgical instrument control device.

Removal of the control device may be performed in while operating the teleoperated surgical system (e.g., hot-swapping control schemes) or while the teleoperated surgical system is not in operation (e.g., shut down, standby, between surgeries, between operations of a single surgery, etc.). The surgical instrument control device may be configured to revert to a previous control scheme (e.g., the control scheme in use before the control shell was installed, a default control scheme) when the control shell is disconnected or removed. In some cases, the control information provided by the control shell includes instructions for removing and/or disconnecting the control shell.

FIG. 15A is a flowchart of a method 1500 of operating a teleoperated surgical system according to some embodiments.

In FIG. 15A, the flowchart shows the method 1500 for connecting the control shell to the surgical instrument control device. FIG. 15B shows a communication diagram corresponding to the method 1500 of FIG. 15A according to some embodiments.

As discussed above, a hardware configuration that communicatively connects the control shell to the surgical instrument control device may be direct or indirect.

Accordingly, while FIGS. 15A-15B describe direct signaling between the control shell and the surgical instrument control device, it will be appreciated that other embodiments (e.g., indirect signaling between the control shell and the surgical instrument control device via a second system or intervening interface) may be conceived without deviating from the gist of the disclosure.

At 1505, the control shell receives a connection query from the surgical instrument control device. The connection query may be transmitted from the surgical instrument control device based on one or more conditions. In some embodiments, the surgical instrument control device transmits the connection query in response to an interrupt process triggered by a hardware connection (e.g., the control shell directly to the surgical instrument control device, the control shell indirectly to the surgical instrument control device). In some embodiments, the surgical instrument control device transmits the connection query upon startup of the surgical instrument control device (e.g., part of a boot up procedure, startup of an application on the surgical instrument control device). In some embodiments, the surgical instrument control device transmits the connection query in a periodic polling process.

At 1510, the control shell transmits a connection message to the surgical instrument control device, in response to the connection query.

At 1515, the control shell receives a configuration query from the surgical instrument control device, in response to the connection message.

At 1520, the control shell transmits a configuration message to the surgical instrument control device, in response to the configuration query. The configuration message includes one or more informational elements that relate to the configuration of the control shell and/or the process of integrating the control shell with a surgical instrument control device. For example, in some embodiments, the configuration message includes the control information, as described above with respect to 1405.

In some embodiments, the configuration message may include shell version information (e.g., number, designation, any appropriate identifier) that corresponds to a predetermined mapping of functions for the control shell and the surgical instrument control device. For example, a plurality of different control shell configurations may be used interchangeably with a surgical instrument control device (e.g., different shell control elements, different control layouts, different procedure-specific considerations), each control shell having a specific version information.

In some embodiments, the shell version information of a given control shell may correspond to specific instructions related to the particular configuration of the given control shell. For example, the shell version information may correspond to a mapping of functions that are configured to the shell control elements and/or procedure-specific operations of the given control shell. When a memory of the control shell includes control information for a plurality of control shell configurations, the shell version information may be used to select the appropriate control information and instructions for integrating the control shell with the preexisting control scheme.

In some embodiments, the configuration message may include a user interface asset and instructions for updating a user interface of the surgical instrument control device to include the user interface asset. A user interface asset may include an indicator/icon for a control element (e.g., a button press indicator, an icon for a function), a condition indicator (e.g., a safety condition indicator, instrument roll position indicator, instrument status indicator, an icon/indicator for an error mode, or discrepancy between control shell and surgical instrument control device), or any appropriate graphical asset that may be used by the control shell or surgical instrument control device.

As described in further detail below with respect to FIGS. 17A-17B, the reverse exchange may also be configured. The configuration message may include instructions for the surgical instrument control device to provide the control shell with a user interface asset and instructions for updating a user interface of the control shell (e.g., for use on a display or visual indicator of the control shell).

After receiving the configuration message, the surgical instrument control device utilizes the content of the configuration message (e.g., the control information) to modify a preexisting control scheme stored therein to include account for the addition of the control shell. The modifications to the preexisting control scheme may include any number of changes. In some embodiments, the modification includes a remapping of functions (e.g., reassignment, recalibration, enabling/disabling) to the new combination of preexisting control elements and shell control elements. In some embodiments, the modification includes the addition and/or removal of functions (e.g., add control input(s) and functions for a second system that is separate from the manipulator assembly and that operates in coordination with the manipulator assembly, disable or remove obsolete or conflicting functions).

Once the surgical instrument control device is modified to account for the integration of the control shell, the surgical instrument control device may transmit a completion message or notification to the control shell. The completion message or notification may indicate that configuration is complete and the surgical instrument control device is ready to receive command inputs and/or instructions from the control shell.

In some embodiments, the surgical instrument control device uses a connection query (e.g., repeating transmission of the connection query at 1505) to determine whether or not the control shell has been removed or disconnected from the surgical instrument control device. For example, the surgical instrument control device may periodically transmit a connection query in a periodic polling process (after modifying the preexisting control scheme) or in response to an interrupt process triggered by a change in hardware connection. When the control shell is still connected to the surgical instrument control device, the control shell may respond to the connection query with a connection message (e.g., the connection message of 1510, a different connection message to skip further configuration messaging) or may provide any appropriate indication of a connection. In some embodiments, the connection query for determining whether or not the control shell has been removed or disconnected does not trigger further configuration queries or messages.

When the surgical instrument control device determines the control shell has been removed or disconnected (e.g., no connection message received after connection query, any appropriate indication of no connection), the surgical instrument control device may use the control information from the control shell (or instructions in the preexisting control scheme) to appropriately modify the current control scheme. For example, the surgical instrument control device may revert to a previous control scheme and/or execute any number of actions and/or instructions (e.g., emergency stop procedures, procedures for changing equipment). In some embodiments, the surgical instrument control device may receive an indication from the control shell to revert to a previous control scheme and/or execute any number of actions and/or instructions prior to removal or disconnection of the control shell (e.g., input from the operator to execute a procedure for disconnecting the control shell).

FIG. 16A is a flowchart of a method 1600 of operating a teleoperated surgical system according to some embodiments.

In FIG. 16A, the flowchart shows the method 1600 for managing communication between the control shell and the rest of the teleoperated surgical system. FIGS. 16B-16C show communication diagrams corresponding to the method 1600 of FIG. 16A according to some embodiments.

Different communication modes may be used to manage active communication between the control shell and the surgical instrument control device. In some embodiments, a first communication mode includes configuring the control shell to transmit an input from a shell control element. For example, an input signal (e.g., an encoder value, a voltage/current signal, a Boolean value, etc.) corresponding to activation of or interaction with the shell control element may be directly passed to the surgical instrument control device and/or a second system that is separate from the manipulator assembly and that operates in coordination with the manipulator assembly. In some embodiments, a second communication mode includes configuring the control shell to process the input from the shell control element into an instruction and subsequently transmit the instruction. For example, an input signal corresponding to activation of or interaction with the shell control element may be processed by the control shell before being passed to the surgical instrument control device and/or a second system that is separate from the manipulator assembly and that operates in coordination with the manipulator assembly.

At 1605, a communication mode of the control shell is determined. In some embodiments, the communication mode is determined by the control shell (e.g., predetermined from a plurality of modes for a given control shell version, the only mode programmed in the control shell). In some embodiments, the communication mode is determined or selected by an operator of the control shell.

When the control shell determines the first communication mode is active, the process continues to 1610. When the control shell determines the second communication mode is active, the process continues to 1620.

At 1610, the control shell transmits the input from the shell control element.

At 1620, the control shell processes the input from the shell control element into an instruction.

At 1625, the control shell transmits the instruction.

FIG. 17A is a flowchart of a method 1700 of operating a teleoperated surgical system according to some embodiments.

In FIG. 17A, the flowchart shows the method 1700 for the control shell to obtain assets and/or information from the surgical instrument control device. FIG. 17B shows a communication diagram corresponding to the method 1700 of FIG. 17A according to some embodiments.

As discussed above with respect to FIGS. 15A-15B, the control shell may be configured to provide assets and/or information to the surgical instrument control device to facilitate modifying a preexisting control scheme. Accordingly, the reverse exchange is also possible to facilitate modifying the control shell to integrate with the surgical instrument control device.

At 1705, the control shell transmits a configuration message including control information to the surgical instrument control device. The control information includes instructions for the surgical instrument control device to transmit an asset and/or information to the control shell.

At 1710, the control shell receives the assets and/or information from the surgical instrument control device. In some embodiments, an asset from the surgical instrument control device may include an indicator/icon for a control element (e.g., a button press indicator, an icon for a function), a condition indicator (e.g., a safety condition indicator, an icon/indicator for an error mode or discrepancy between control shell and surgical instrument control device), or any appropriate graphical asset that may be used by the control shell. In some embodiments, the information from the surgical instrument control device may include instructions for use of the asset by the control shell (e.g., for use on a display or visual indicator of the control shell).

At 1715, the control shell utilizes the assets and/or information during operation of the teleoperated surgical system.

FIG. 18 is a flowchart of a method 1800 of operating a teleoperated surgical system according to some embodiments.

In FIG. 18, the flowchart shows the method 1800 for enabling or disabling a function based on shell control element that includes a locking function. For example, as shown in FIGS. 6A-6B, one or more embodiments of the shell control element include a locking function (e.g., a lock component disposed on the body of the control shell and a key component that is removable from the lock component). As discussed above with respect to FIG. 15A-15B, the control information may include calibration information for enabling or disabling a function of the control shell or the surgical instrument control device based on the locking function (e.g., a state of the key component).

At 1805, the control shell determines whether or not the locking function of the shell control element is enabled or disabled. For example, in the case of a removable cover as the key component and a base portion as the lock component as shown in FIGS. 6A-6B, the locking function of the shell control element is considered disabled (i.e., unlocked) when the key component is connected to the lock component (i.e., the removable cover is installed onto the base portion). Likewise, the locking function of the shell control element is considered enabled (i.e., locked) when the key component is disconnected from the lock component (i.e., the removable cover is removed from the base portion).

In some embodiments, whether or not the locking function is enabled or disabled may be determined by a sensor or detector.

In some embodiments, control information corresponding to the shell control element with the locking function may include calibration information for determining whether or not the locking function is enabled or disabled. In other words, a locking function of the control shell or the surgical instrument control device based on an input to or interaction with the shell control element.

While a limited number of embodiments have been described in the present disclosure, it will be appreciated that other embodiments (e.g., a keycard and proximity detector, a key and lock, a biometric feature of an operator and a biometric lock) may be devised without departing from the gist of the invention.

When the control shell determines the locking function is disabled (e.g., the key component is connected to the lock component), the process continues to 1810. When the control shell determines the locking function is enabled (e.g., the key component is not connected to the lock component), the process continues to 1820.

At 1810, the function of the control shell or the surgical instrument control device is enabled. In some embodiments, a function of a second system, that is separate from the manipulator assembly and that operates in coordination with the manipulator assembly, is enabled.

At 1820, the function of the control shell or the surgical instrument control device is disabled. In some embodiments, a function of a second system, that is separate from the manipulator assembly and that operates in coordination with the manipulator assembly, is disabled.

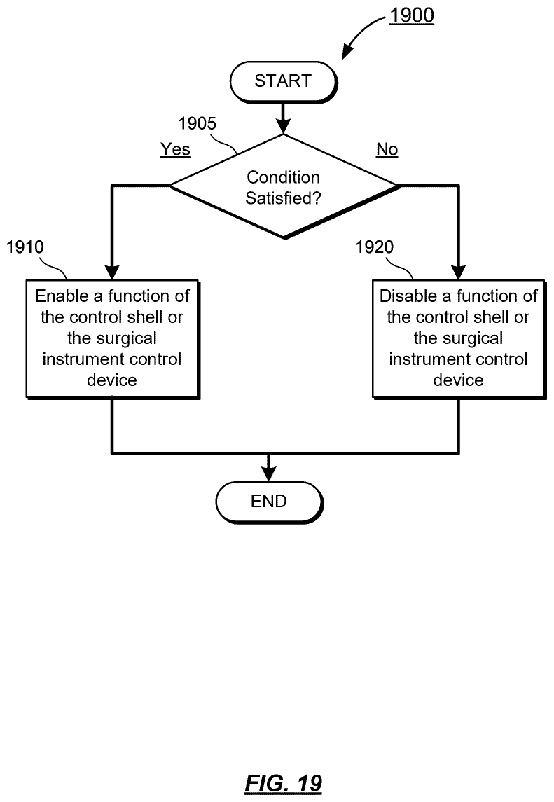

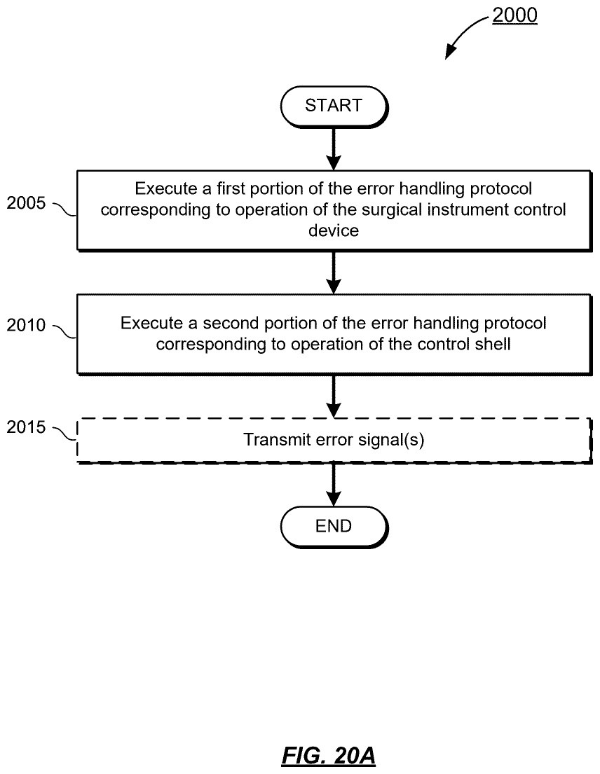

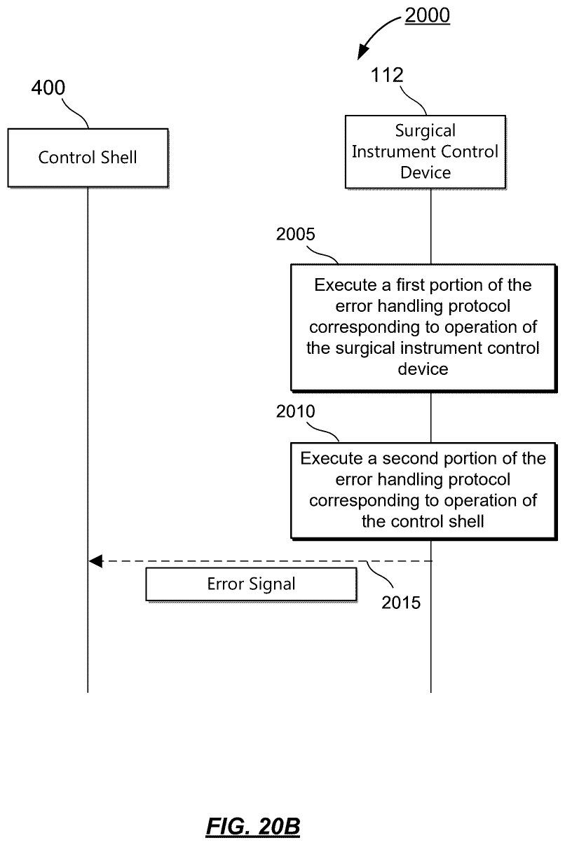

FIG. 19 is a flowchart of a method 1900 of operating a teleoperated surgical system according to some embodiments.

In FIG. 19, the flowchart shows a method 1900 for enabling or disabling a function based on shell control element that includes a touch sensor. For example, as shown in FIGS. 7A-7B, one or more embodiments of the shell control element include a touch sensor (e.g., a touch screen display, a touch interface, a contact or proximity sensor). As discussed above with respect to FIG. 15A-15B, the control information may include calibration information for enabling or disabling a function of the control shell or the surgical instrument control device based on the touch sensor (e.g., an input signal from, interaction with, activation of).

At 1905, the control shell determines whether or not a condition of the touch sensor is satisfied. For example, to avoid in advertent activation during a specific procedure, a function may be disabled when the touch sensor detects an stray movement from the operator. In another example, a function may be enabled when detecting interaction from the operator (e.g., operator intentionally activates the touch sensor).

In some embodiments, control information corresponding to the touch may include calibration information for one or more conditions. In other words, a function of the control shell or the surgical instrument control device (or a second system) may be activated or changed based on an input to or interaction with the touch sensor.

When the control shell determines the condition is satisfied, the process continues to 1910. When the control shell determines the condition is not satisfied, the process continues to 1820.

At 1910, the function of the control shell or the surgical instrument control device is enabled. In some embodiments, a function of a second system, that is separate from the manipulator assembly and that operates in coordination with the manipulator assembly, is enabled.

At 1920, the function of the control shell or the surgical instrument control device is disabled. In some embodiments, a function of a second system, that is separate from the manipulator assembly and that operates in coordination with the manipulator assembly, is disabled.

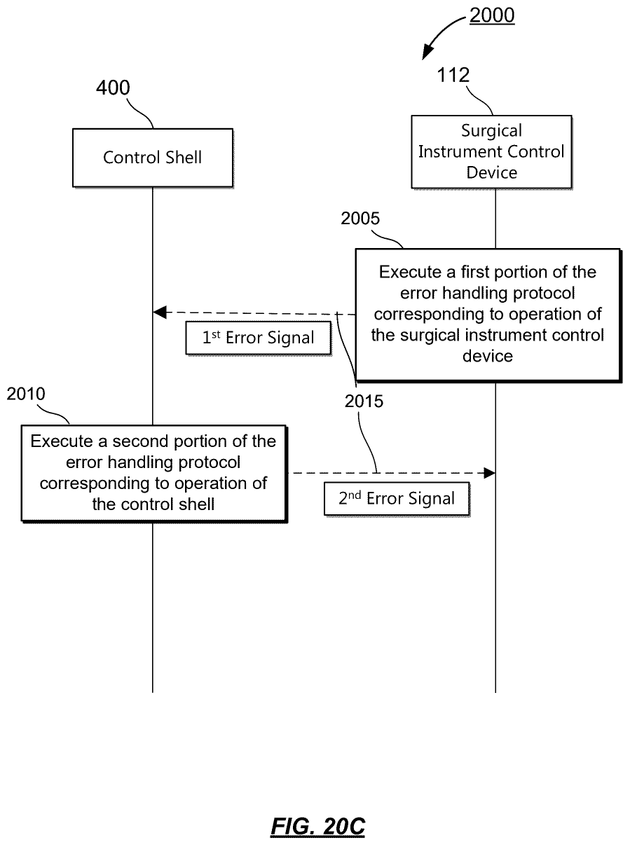

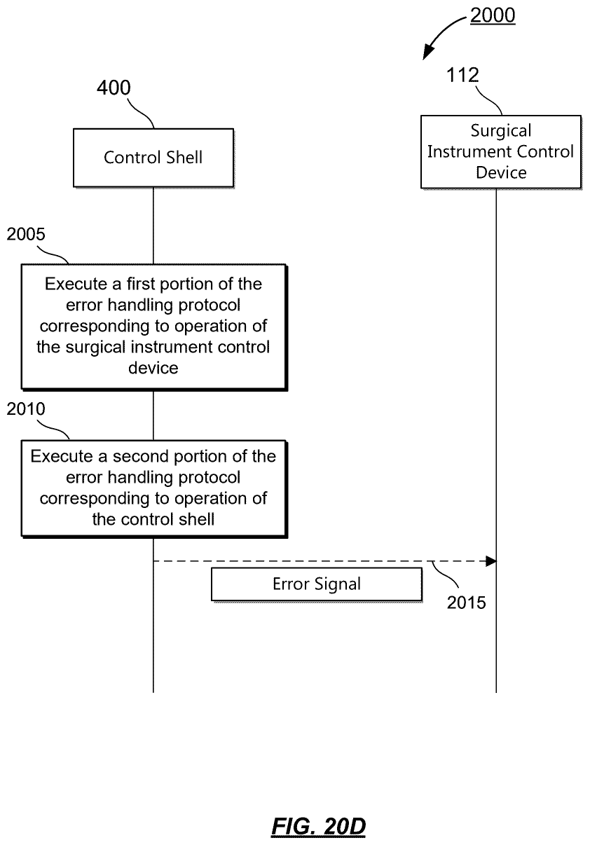

FIG. 20A is a flowchart of a method of operating a teleoperated surgical system according to some embodiments.

In FIG. 20A, the flowchart shows the method 2000 for handling errors in the teleoperated surgical system. FIGS. 20B-20D show communication diagrams corresponding to the method 2000 of FIG. 20A according to some embodiments.

The modifying of the preexisting control scheme includes creating an error handling protocol for the surgical instrument control device and the control shell. In some embodiments, the configuration message and/or control information transmitted from the control shell to the surgical instrument control device may include instructions that define how to identify and respond to errors in the control shell. For example, an error handling protocol may include signal readout checks of shell control element inputs (e.g., comparing signals from multiple encoders on a single input), hardware health checks (e.g., fault checks, safety checks), software health checks (e.g., firmware checks), or any appropriate diagnostic test or procedure.

In some embodiments, the error handling protocol for the teleoperated surgical system is divided into two portions: a first portion corresponding to operation of the surgical instrument control device; and a second portion corresponding to operation of the control shell. Accordingly, the configuration message and/or control information transmitted from the control shell may define which component of the teleoperated surgical system, the surgical instrument control device or the control shell, executes error handling for the respective components of the teleoperated surgical system.

At 2005, the surgical instrument control device or the control shell executes a first portion of the error handling protocol corresponding to operation of the surgical instrument control device.

In some embodiments, as shown in FIGS. 20B-20C, the surgical instrument control device executes the first portion of the error handling protocol. In other words, the surgical instrument control device may be configured to monitor and manage its own errors according to the preexisting control scheme.

In some embodiments, as shown in FIG. 20D, the control shell executes the first portion of the error handling protocol. In other words, the preexisting control scheme executed by the surgical instrument control device may be modified by offloading error handling from the surgical instrument control device to the control shell (e.g., by instructions transferred from the surgical instrument control device to the control shell).

At 2010, the surgical instrument control device or the control shell executes a second portion of the error handling protocol corresponding to operation of the control shell.

In some embodiments, as shown in FIGS. 20C-20D, the control shell executes the second portion of the error handling protocol. In other words, the control shell may be configured to monitor and manage its own errors according to the control scheme install in a memory of the control shell.

In some embodiments, as shown in FIG. 20B, the surgical instrument control device executes the second portion of the error handling protocol. In other words, the preexisting control scheme executed by the surgical instrument control device may be modified by adding error handling protocols for the control shell (e.g., by instructions transferred from the control shell to the surgical instrument control device).

Optionally, at 2015, the surgical instrument control device and/or the control shell transmits one or more error signals. An error signal may include information (e.g., error codes), instructions (e.g., commands to execute), assets (e.g., error/warning images or indicators), control scheme information (e.g., remapping information such as safety control lockouts) corresponding to an error. The first and/or the second portions of the error handling protocol may include instructions for transmitting an error signal to inform another component of the teleoperated surgical system of the error.

In some embodiments where the first and the second portions of the error handling protocol are independent of each other, distributing error signals in the teleoperated surgical system ensures the appropriate action(s) are taken to resolve the error and mitigate any potential risks associated with the error.

Returning to the non-limiting example of a teleoperated surgical system shown in FIGS. 11A-11B, the methods of FIG. 15A-20D may be applied to using a control shell to control a fluid system that is separate from the manipulator assembly and that operates in coordination with the manipulator assembly.

In some embodiments, the surgical instrument control device may control a manipulator assembly to position the input/output of the fluid system while the control shell includes a plurality of shell control elements to control the fluid system itself. For example, the control shell may include cutout portions disposed to allowed preexisting control elements (e.g., the insertion/retraction control, the steering control, and the emergency stop button) of the surgical instrument control device to protrude through the control shell and remain accessible to the operator. Furthermore, the control shell includes a plurality of toggleable shell control elements (e.g., buttons) to control functions of the manipulator (e.g., movement, positioning) and/or fluid system (e.g., insufflation, suction, lens wash/clean, irrigation). In some embodiments, the control shell may include a foot pedal connected to the control shell by a wired or wireless connection interface.

The hardware configuration of the control shell for the fluid system is reflected in control information stored in a memory of the control shell. For example, the control information may include a routing table that associates each togglable shell control element with one or more fluid control elements (e.g., activating valves, pumps, etc., controlling a sequence of actions) of the fluid system. The routing table may include any number of associations and may be customizable to the specific functions and capabilities of the fluid system. Accordingly, when the control information is transmitted to the surgical instrument control device, a preexisting control scheme is modified to create a new control scheme that includes: a first mapping of control elements of the surgical instrument control device to functions of the manipulator assembly; and a second mapping of the shell control element(s) of the control shell to a function of the fluid system.

Although the disclosure has been described with respect to a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A control shell that is an accessory to a surgical instrument control device of a teleoperated surgical system, the control shell comprising:
a body, formed to overlay onto a user input console of the surgical instrument control device, that includes:
a cutout region disposed to allow a first preexisting control element of the surgical instrument control device to remain accessible through the control shell to an operator of the surgical instrument control device;
a shell control element for controlling a function of the teleoperated surgical system; and
a communication interface;
a memory that includes instructions for integrating the shell control element into the surgical instrument control device and controlling robotic movement of an instrument; and
a processor configured to:
communicate with the surgical instrument control device via the communication interface; and
integrate the shell control element into the surgical instrument control device based on the instructions stored in the memory.

2. The control shell of claim 1, wherein
the shell control element controls a degree of freedom of a manipulator assembly of the teleoperated surgical system that is not controlled in the surgical instrument control device, and
the shell control element include a single degree of freedom that controls a roll motion of an endoscope of the manipulator assembly.

3. The control shell of claim 1, wherein
the shell control element controls a system that is separate from a manipulator assembly of the teleoperated surgical system and that operates in coordination with the manipulator assembly.

4. The control shell of claim 3, wherein
the shell control element controls a fluid system connected to the manipulator assembly,
the shell control element is a first control element to control emitting one or more fluids from the manipulator assembly, and
the control shell further includes a second control element to control evacuation of one or more fluids by the manipulator assembly.

5. The control shell of claim 4, wherein
the control shell includes a foot pedal as a third control element to control an irrigation pump connected to the manipulator assembly.

6. The control shell of claim 1, wherein
the shell control element modifies a preexisting function of the surgical instrument control device, and
based on the instructions stored in the memory, the processor is configured to modify a preexisting control element of the surgical instrument control device.

7. The control shell of claim 6, wherein
based on the instructions stored in the memory, the processor is configured to remap the preexisting control element of the surgical instrument control device to control a different function of the teleoperated surgical system.

8. The control shell of claim 1, wherein
the shell control element controls an energy system that is separate from a manipulator assembly of the teleoperated surgical system and that operates in coordination with the manipulator assembly, and
the shell control element controls a mode or an operation of the energy system.

9. The control shell of claim 1, wherein
the shell control element controls an imaging system that is separate from a manipulator assembly of the teleoperated surgical system and that operates in coordination with the manipulator assembly, and
the shell control element controls a mode or an operation of the imaging system.

10. A teleoperated surgical system including:
a surgical instrument control device;
a control shell that is an accessory to the surgical instrument control device,
wherein the control shell comprises:
a body, formed to overlay onto a user input console of the surgical instrument control device, that includes:
a cutout region disposed to allow a first preexisting control element of the surgical instrument control device to remain accessible through the control shell to an operator of the surgical instrument control device;
a shell control element for controlling a function of the teleoperated surgical system; and
a communication interface;
a memory that includes instructions for integrating the shell control element into the surgical instrument control device and controlling robotic movement of an instrument; and
a processor configured to:
communicate with the surgical instrument control device via the communication interface; and
integrate the shell control element into the surgical instrument control
device based on the instructions stored in the memory.

11. A method of operating a teleoperated surgical system including a control shell that is an accessory to a surgical instrument control device, the method comprising:
transmitting, from the control shell to the surgical instrument control device, control information corresponding to a shell control element of the control shell, wherein the control information includes a correlation between a function of the teleoperated surgical system and the shell control element;
modifying a preexisting control scheme stored in a preexisting input device to include the control information, the preexisting control scheme comprising instructions for controlling a first robotic movement of a first instrument according to a first user input received at the surgical instrument control device, the control information comprising instructions for controlling a second robotic movement of a second instrument according to a second user input received at the shell control element; and
initializing the teleoperated surgical system to receive and process both the second user input from the shell control element and the first user input from the surgical instrument control device.

12. The method of claim 11, further comprising:
in response to an input at the shell control element, transmitting, from the control shell, the input or an instruction corresponding to the input; and
operating the teleoperated surgical system based on the input or the instruction

13. The method of claim 11, further comprising:
receiving, by the control shell from the surgical instrument control device, a connection query;
transmitting, from the control shell to the surgical instrument control device, a connection message in response to the connection query;
receiving, by the control shell from the surgical instrument control device, a configuration query in response to the connection message; and
transmitting, from the control shell to the surgical instrument control device, a configuration message in response to the configuration query,
wherein the configuration message includes the control information.

14. The method of claim 11, wherein
the control information corresponds to a second system that is separate from a manipulator assembly and that operates in coordination with the manipulator assembly, and
the modifying of the preexisting control scheme creates a new control scheme that includes:
a first mapping of control elements of the preexisting input device to functions of the manipulator assembly; and
a second mapping of the shell control element of the control shell to a function of the second system.

15. The method of claim 14, wherein
the second system is a fluid system connected to the manipulator assembly, and
the control shell includes a plurality of shell control elements,
the second mapping includes:
a mapping of a first shell control element to control fluid emission from the fluid system connected to the manipulator assembly, and
a mapping of a second shell control element to control fluid evacuation by the fluid system connected to the manipulator assembly.

16. The method of claim 11, wherein
the modifying of the preexisting control scheme further includes remapping a preexisting control element of the surgical instrument control device.

17. The method of claim 11, wherein
the modifying of the preexisting control scheme includes creating an error handling protocol for the surgical instrument control device and the control shell based on the control information.

18. The method of claim 17, further comprising:
executing, by the surgical instrument control device, a first portion of the error handling protocol corresponding to operation of the surgical instrument control device;
executing, by the surgical instrument control device, a second portion of the error handling protocol corresponding to operation of the control shell; and
transmitting an error signal from the surgical instrument control device to the control shell.

19. The method of claim 17, further comprising:
executing, by the surgical instrument control device, a first portion of the error handling protocol corresponding to operation of the surgical instrument control device; and
executing, by the control shell, a second portion of the error handling protocol corresponding to operation of the control shell,
transmitting, as part of the first portion of the error handling protocol, a first error signal from the surgical instrument control device to the control shell; and
transmitting, as part of the second portion of the error handling protocol, a second error signal from the control shell to the surgical instrument control device,
wherein the first and the second portions of the error handling protocol are independent of each other.

20. The method of claim 17, further comprising:
executing, by the control shell, a first portion of the error handling protocol corresponding to operation of the surgical instrument control device; and
executing, by the control shell, a second portion of the error handling protocol corresponding to operation of the control shell; and
transmitting an error signal from the control shell to the surgical instrument control device.
